(19) United States
(12) Patent Application Publication
(10) Pub. No.: US 2003/0219830 A1
(43) Pub. Date: Nov. 27, 2003

(54) Methods of evaluating glycomolecules for enhanced activities

(76) Inventors: Ganesh Venkataraman, Bedford, MA (US); Zachary Shriver, Boston, MA (US); Ram Sasisekharan, Cambridge, MA (US)

Correspondence Address: FISH & RICHARDSON PC, 225 FRANKLIN ST, BOSTON, MA, 02110, US

(21) Appl. No.: 10/388,868

(22) Filed: Mar. 14, 2003

Related U.S. Application Data
(63) Continuation-in-part of application No. 10/244,805, filed on Sep. 16, 2002.
(60) Provisional application No. 60/322,232, filed on Sep. 14, 2001.

(51) Int. Cl. G01N 33/53; C08B 37/00; C12P 21/06; C07K 14/435

(52) U.S. Cl. 435/7.1; 435/68.1; 536/53; 530/395

(57) ABSTRACT

Methods to evaluate and rapidly produce and identify polysaccharides, and other sugar structures, having enhanced activities, have been developed. The methods include producing a molecule, e.g., a therapeutic molecule, which includes a first, non-saccharide moiety (e.g., a protein, polypeptide, peptide, amino acid or lipid) and a second, saccharide, moiety. The method includes: determining the chemical composition and structure of all or a portion of the second, saccharide moiety, and evaluating or screening the molecule, e.g., for a biological activity or other chemical or physical property. In some embodiments, the step of determining the chemical structure and composition of the second moiety includes a comparison of one or more properties of the second moiety with a database, e.g., a database which correlates such one or more properties with structure or function of a polysaccharide.

### RELATED APPLICATIONS

This application is a continuation-in part of, and claims priority to, U.S. Ser. No. 10/244,805, filed Sep. 16, 2002, which claims priority to U.S. provisional application No. 60/322,232 filed on Sep. 14, 2001, the contents of which are incorporated herein by reference.

### BACKGROUND OF THE INVENTION

Glycomics is the study of sugars, information dense molecules that occur, in both linear and branched forms, in isolated form, as a structure on a cell or organelle, or on molecules such as proteins (referred to as glycoproteins) or lipids (referred to as glycolipids). Linear sugars are found on cell surfaces, attached to proteins and lipids and provide characteristic cellular signatures, mediate cell-cell communications, and actively orchestrate intracellular signal transduction. Branched sugars are found on protein surfaces, among other biopolymers, and provide characteristic protein signatures, mediate protein localization and targeting, and actively modulate protein efficacy, stability pharmacokinetics, and/or therapeutic (clinical) potency.

Although the importance of polysaccharides and other sugars has been recognized, the biotechnology field has not focused on these structures, largely due to the lack of technology enabling such a focus, and has not developed methods for automated sequencing, synthesis, or screening for biological activities.

### SUMMARY OF THE INVENTION

Methods to rapidly produce and identify saccharides, and other sugar structures, associated with glycomolecules having enhanced activities have been developed. The methods include some or all, typically all of the following: determining the chemical composition and/or structure of a saccharide moiety, e.g., a saccharide moiety having a defined activity, e.g., a polysaccharide moiety having a defined activity, to analyze the sequence of sugars on molecules such as proteins, polypeptides and lipids, modifying the chemical composition or structure of the saccharide moiety, using for example enzymatic or solid-phase methods, and screening the modified saccharide moiety as part of a glycomolecule, for altered activity of the glycomolecule. Preferably, multiple features including structure, composition, and reactivity of the polysaccharide moiety are determined. The information obtained can then be used to, e.g., synthesize saccharide, e.g., polysaccharide moieties of interest using, e.g., enzymatic, chemical, or chemoenzymatic synthesis, or to alter the prevalence of a selected moiety or moieties in a heterogeneous population of molecules, e.g., a population of glycomolecules having a plurality of polysaccharide moieties. As used herein, the term “plurality” refers to two or more. In addition, the structure or composition can optionally be modified, and then re-screened for altered activity of a glycomolecule such as a glycoprotein, proteoglycan, glycopeptide or glycolipid.

Accordingly, in one aspect, the invention features a method for producing a glycomolecule, e.g., a therapeutic molecule, which includes at least a first, non-saccharide moiety (e.g., a protein, polypeptide, peptide, amino acid or lipid) and a second, polysaccharide moiety. The method includes: determining the chemical composition and structure of all or a portion of the second moiety, modifying the structure of the second moiety to provide a modified second moiety, and evaluating or screening the glycomolecule having the modified second moiety, e.g., for a biological activity or other chemical or physical property. In some embodiments, the step of determining the chemical structure and composition of the second moiety includes a comparison of one or more properties of the second moiety with a database, e.g., a database which correlates such one or more properties with structure or function of a saccharide, e.g., polysaccharide.

In some embodiments, the second saccharide moiety has a defined activity, e.g., an activity defined by comparison to a database of known saccharides, and the evaluation or screening includes evaluating the molecule for altered, enhanced or optimized biological activity of the modified second moiety.

In some embodiments, the chemical structure and composition of the second saccharide moiety, e.g., a polysaccharide moiety, can be determined by comparing the length and/or molecular mass of the second moiety to a database of saccharides, e.g., polysaccharides, having known length and/or molecular mass; selecting from the database a subpopulation of known polysaccharides having the same length or a similar molecular mass as the second moiety; applying an experimental constraint to the second moiety to determine a property of the second moiety; comparing the property of the second moiety to the subpopulation; and eliminating from the subpopulation saccharides which do not have the property of the second moiety when subjected to the same experimental constraint. This process can be repeated one or more times, e.g., using a different experimental constraint, to eliminate additional saccharides from the subpopulation.

Experimental constraints can include, but are not limited to: enzymatic digestion, e.g., with an exoenzyme, an endoenzyme, chemical digestion, chemical modification, chemical peeling, interaction with a binding compound, and enzymatic modification, e.g., sulfonation at a particular position. Examples of enzymes which can be used to digest a polysaccharide moiety include α-galactosidase to cleave a α1→3 glycosidic linkage after a galactose, β-galactosidase to cleave a β1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose, β-N-Acetylhexosaminidase to cleave non-reducing terminal β1→2,3,4,6 linked N-acetylglucosamine, and N-acetylgalactosamine, alpha-N-Acetylgalactosaminidase to cleave terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

Properties of the saccharide which can be determined include: the mass of part or all of the oligosaccharide structure, the charges of the chemical units of the saccharide, identities of the chemical units of the saccharide, confirmations of the chemical units of the saccharide, total charge of the saccharide, total number of sulfates of the saccharide, total number of acetates, total number of phosphates, presence and number of carboxylates, presence and number of aldehydes or ketones, dye-binding of the saccharide, compositional ratios of substitutents of the saccharide, compositional ratios of anionic to neutral sugars, presence of uronic acid, enzymatic sensitivity, linkages between chemical units of a polysaccharide, charge, branch points, number of branches, number of chemical units in each branch, core structure of a branched or unbranched saccharide, the hydrophobicity and/or charge/charge density of each branch, absence or presence of GlcNAc and/or fucose in the core of a branched polysaccharide, number of mannose in an extended core of a branched polysaccharide, presence or absence of sialic acid on a branched chain of a polysaccharide, the presence or absence of galactose on a branched chain of a polysaccharide.

In some embodiments, the method includes using the determined composition and structure of the second moiety to produce the modified second moiety or a portion thereof using enzymatic, chemical, or chemoenzymatic synthesis, or any combination thereof. In other embodiments, the modification of the second moiety includes using the determined composition and structure of the second moiety to produce the modified second moiety or portion thereof using metabolic engineering or any combination of the above.

The modification of the second moiety can include, e.g., changing one or more of the identity, number, or linkage of one or more chemical units in the second moiety, or changing the prevalence of a selected second moiety in a population of molecules, which includes glycomolecules. For instance, in some embodiments, the modification includes changing the number of branches in the second moiety. A polysaccharide moiety can be modified, e.g., by removing one or more branches from a polysaccharide (e.g., an endoglycan such as EndoF2 can be used to remove a branch from a biantennary polysaccharide) or adding one or more branches to a polysaccharide moiety (e.g., a core α1→6 fucose or β1→4 GlcNAc can be added to a polysaccharide moiety). Additional monosaccharides can be added to the additional branch or branches of a modified polysaccharide moiety.

In another embodiment, the saccharide, e.g., polysaccharide, moiety is enzymatically modified, e.g., by enzymatic cleavage and/or enzymatic addition of one or more chemical units.

In one embodiment, a polysaccharide moiety can be modified by enzymatically removing one or more chemical unit(s) of the polysaccharide, e.g., one or more of a sialic acid, fucose, galactose, glucose, xylose, GlcNAc, and/or a GalNAc can be removed from the polysaccharide moiety. Examples of enzymes which can be used to remove a chemical unit from the polysaccharide moiety include: α-galactosidase to cleave a α1→3 glycosidic linkage after a galactose, β-galactosidase to cleave a β1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose, a N-acetylglucosiaminidase to cleave a β1→2, a β1→4 or β1→6 linkage after a GlcNAc.

In another embodiment, a saccharide moiety, e.g., polysaccharide moiety, can be modified by enzymatically adding one or more chemical unit(s) to the saccharide, e.g., one or more of a sialic acid, fucose, galactose, glucose, xylose, GlcNAc, and/or a GalNAc can be added to the saccharide moiety. Examples of enzymes which can be used to add a chemical unit include: sialyltransferase, e.g., α2→3 sialyltransferase or α2→6 sialyltransferase, fucosyltransferase, e.g., α1→2 fucosyltransferse, α1→3 fucosyltransferase, α1→4 fucosyltransferase or α1→6 fucosyltransferase, galactosyltransferase (e.g., α1→3 galactosyltransferase, β1→4 galactosyltransferase or β1→3 galactosyltransferase) and a N-acetylglucosaminyltransferase (e.g., N-acetylglucosaminyltransferase I, II or III).

In other embodiments, a saccharide moiety, e.g., polysaccharide moiety, can be modified by removing one or more chemical units and adding one or more chemical units to the saccharide moiety. In another embodiment, the saccharide can be modified by altering one or more substituent associated with the saccharide, e.g., associated with a chemical unit of a saccharide. For example, sulfonation, e.g., of a sialic acid, can be modified to add a sulfate, e.g., using a sulfatransferase, or by removing a sulfate, e.g., a sulfatase.

In other embodiments, a saccharide moiety can be modified by removing the entire saccharide moiety from the glycomolecule.

In another embodiment, the modification of the saccharide moiety, e.g., the polysaccharide moiety, can be effected by altering a synthetic process which produces a saccharide moiety, e.g., by adding an excess of a substrate or intermediate in a synthetic reaction. For example, one or more of a sialic acid, fucose, galactose, glucose, xylose, GlcNAc, and/or a GalNAc can be added to the saccharide moiety by adding one or more of these monosaccharides, e.g., activated forms of these monosaccharides or precursors to these monosaccharides, to a cell, e.g., a recombinant cell which produces the saccharide to be modified. In addition, an enzyme which incorporates a chemical unit into a saccharide chain can be added. Examples of enzymes which can be used to add a chemical unit include: sialyltransferase, e.g., α2→3 sialyltransferase or α2→6 sialyltransferase, fucosyltransferase, e.g., α1→2 fucosyltransferse, α1→3 fucosyltransferase, α1→4 fucosyltransferase or α1→6 fucosyltransferase, galactosyltransferase (e.g., α1→3 galactosyltransferase, β1→4 galactosyltransferase or β1→3 galactosyltransferase) and a N-acetylglucosaminyltransferase (e.g., N-acetylglucosaminyltransferase I, II or III). In other embodiments, an additional agent can be used to increase incorporation of a chemical unit in a polysaccharide. For example, a monosaccharide can be peracetylated to increase diffusion of the monosaccharide into a cell, e.g., a recombinant cell. In other embodiments, the agent can decrease or eliminate the presence of an enzyme present in the cell (e.g., UDP-N-acetylglucosamine-2-epimerase) such that increased incorporation of the monosaccharide units can occur.

In some embodiments, the modification is effected by directly modifying a saccharide moiety naturally present on the first, non-saccharide, moiety, thereby providing a modified second moiety. In other embodiments, the modification is effected by attaching a second saccharide moiety which differs from an existing saccharide naturally attached to said first moiety, e.g., by attaching a new or modified saccharide moiety to a first moiety that does not naturally include a second moiety, e.g., a first moiety in which a saccharide naturally attached to the first moiety has been removed, or a first moiety that does not normally have a saccharide attached to it. In other embodiments, the first moiety has an existing saccharide naturally attached to it removed, and a saccharide not naturally attached to it is added as a modified second moiety, e.g., added at a position in the first moiety where the naturally existing saccharide had previously been attached or at a position in the first moiety where no naturally existing saccharide had previously been attached. In other embodiments, a second saccharide moiety is attached to a preselected site on a non-saccharide moiety. In other embodiments, additional saccharide moieties are attached to multiple sites on the non-saccharide moiety; the additional saccharide moieties may be chemically identical or different.

In some embodiments, an activity of the molecule is increased, decreased, eliminated by the modified second moiety. In one embodiment, an activity of the molecule is increased by the modified second moiety and the activity which is increased is selected from the group consisting of improved therapeutic index or activity after clinical administration, half-life, stability, IC₅₀ (ED₅₀), and binding. In another embodiment, an activity of the molecule is decreased or eliminated by the modified second moiety and the activity which is decreased or eliminated is a side effect associated with therapy, e.g., toxicity and/or immunogenicity.

In some embodiments, the first moiety is a protein or fragment thereof and the modified second moiety is an N-linked polysaccharide, e.g., an N-linked polysaccharide selected from the group consisting of simple, complex, hybrid and high mannose polysaccharides. In another embodiment, the first moiety is a protein or fragment thereof and the modified second moiety is an O-linked saccharide. In yet another embodiment, the first moiety is a protein or fragment thereof and there are at least two or more modified second moieties associated with it, e.g., two or more N-linked polysaccharides, two or more O-linked saccharides, or combinations thereof. The protein or fragment thereof can be modified, e.g., by modifying the amino acid sequence to add a site for attaching the second moiety, e.g., the amino acid sequence of the protein or fragment thereof can be modified to replace an amino acid which does not serve as a site for attaching a saccharide or serves as a site for attaching a one type of saccharide (e.g., an O-linked saccharide) with another amino acid which serves as a site for attaching a different type of saccharide (e.g., an N-linked polysaccharide), or by adding to the amino acid sequence an additional amino acid which serves as a site for attaching a saccharide.

In other embodiments, the modified second moiety can be a glycosaminoglycan, or a Lewis sugar. For example, the modified second moiety can be, e g., a Lewis sugar selected from the group consisting of: a type 1 Lewis structure (e.g., Lewis x, Lewis y, sialyl Lewis x. 6-sulfo-sialyl Lewis x) and a type 2 Lewis structure (e.g., Lewis a, Lewis b, sialyl Lewis a and 3′-sulfo-Lewis a).

In some embodiments, the glycomolecule is formed by attaching the first moiety and the modified second moiety by ligation, e.g., chemical, enzymatic or chemoenzymatic ligation.

In another embodiment, the modification includes purifying or enriching for one or more selected molecule species present in a preparation of molecules that includes molecules having a first and second moiety. In other words, the modification can be a property of a collection of molecules, wherein the modification is not the introduction of a new second moiety but the alteration of the amounts or relative amounts of one or more species of a molecule having particular second moiety. For example, one begins with a heterologous population of molecules that includes glycomolecules, which are heterologous in the sense that the structure of the second moiety is heterologous, e.g., a population of a particular first moiety not all of which have the same second saccharide moiety. The structure of one or more of the heterologous second moiety species is determined. The modification can be effected by altering the structure of the second moiety or it can be effected by enriching for or depleting one or more of the heterologous second moiety species. By way of illustration, one can begin with a preparation of a protein, some of the protein molecules of which have a complex polysaccharide second moiety and some of which do not. The preparation can be enriched for proteins having the complex structure of the second moiety using methods described herein.

In another aspect, the invention provides methods for selecting a preparation of a glycomolecule, e.g., a glycoprotein or glycopeptide, having one or more selected activities, e.g., reduced immunogenicity. The method can include some or all, preferably all, of the following: providing a plurality of preparations of a glycomolecule, e.g., a plurality of preparations each produced using a different process, e.g., processes which differ in one or more parameters, e.g., experimental or manufacturing parameters; determining the chemical structure or composition of one or more saccharides associated with the glycomolecule in each of the plurality of preparations; and selecting a preparation of the glycomolecule having one or more saccharides that are correlated with the selected activity. In one embodiment, the method can also include analyzing one or more of the preparations of a glycomolecule to evaluate whether it has the selected activity. The selected activity can be any activity, e.g., altered therapeutic efficacy or profile, e.g., reduced side effects, e.g., reduced immunogenicity.

In another aspect, the invention provides methods for analyzing a preparation of a glycomolecule, e.g., a glycoprotein or glycopeptide, having one or more selected activities, e.g., reduced immunogenicity. The method includes providing a preparation of a glycomolecule made by a first process, evaluating a selected activity of the preparation of the glycomolecule made by the first process, and comparing the chemical structure or composition to the chemical structure or composition of a glycomolecule made by a second, e.g., a different, process. The determination of the chemical structure or composition of the preparation of glycomolecule made by the first process can be performed before or after the selected activity is evaluated. In one embodiment, the selected activity of the preparation of glycomolecule made by the first and second process is known or determined, and when the chemical structure or composition of one or more of the saccharides of the glycomolecule in the preparation made by the first process differs from the chemical structure or composition of one or more of the saccharides of the glycomolecule in the preparation made by the second process (e.g., a corresponding saccharide), that information can be used, e.g., to evaluate a correlation between the differing saccharides and the activity. The method can also include selecting a preparation, e.g., a preparation that demonstrates the selected activity, e.g., reduced immunogenicity, based on the presence or absence of a saccharide. The method can further include using the chemical structure or composition of the glycomolecule that demonstrates the selected activity to evaluate one or more preparations of a glycomolecule, e.g., preparations made by a process or processes that differ from the first and second processes.

In another embodiment, the selected activity of a first preparation of a glycomolecule, e.g., a glycoprotein or glycopeptide, made by a first process is known, and differs from the activity of a second preparation of the glycomolecule made by a second process, and the chemical structure or composition of one or more of the saccharides of the first preparation made by the first process does not differ from the chemical structure or composition of one or more of the saccharides of the second preparation made by the second process, e.g., corresponding saccharides do not differ, and the preparations can be subjected to further analysis to evaluate the cause of the differences in the selected activity.

The different process (e.g., the first and second processes) used to make the preparation can differ, for example, in on ore more of the parameters for producing, purifying, or formulating the preparations.

In some embodiments, the plurality of preparations of glycomolecules differ in glycospecies, e.g., species of the glycomolecule that differ in the chemical structure or composition of the saccharides associated with the glycomolecule, e.g., the presence, absence, position, or number of one or more saccharides. In some embodiments, the preparations can include a number of different glycospecies of the glycomolecule. In some embodiments, the plurality of preparations include a plurality of the same glycospecies at different levels, e.g., each preparation is enriched for a glycospecies that is not enriched in at least one other preparation.

In another aspect, the method includes providing a plurality of preparations of a glycomolecule produced by different processes; determining the chemical structure or composition of one or more saccharides associated with the glycomolecule; analyzing one or more of the preparations of the glycomolecule to evaluate the presence, absence, or level of a selected activity; and correlating the chemical structure or composition of one or more saccharides associated with the glycomolecule with the presence, absence, or level of the activity. In one embodiment, the method includes selecting a preparation having a desired activity and selecting the process which produced that preparation, and performing the selected process to make additional preparations, e.g., batches, of the glycomolecule.

In another aspect, the method includes providing a first preparation of a glycomolecule made by a first process, providing a second preparation of the glycomolecule made by a second process, e.g., a second process different from the first process in one or more parameters, e.g., experimental or manufacturing parameters, evaluating each of the first and second preparation for a selected activity, e.g., therapeutic efficacy or profile, e.g., the presence or absence of a side effect, e.g., immunogenicity; and determining the chemical structure or composition of a saccharide associated with the glycomolecules of the first and second preparations. In some embodiments, the method can also include selecting the preparation having or lacking the selected property. In some embodiments, the method includes selecting the first or second process which produced a preparation having a desired activity, and performing the selected process to make additional preparations, e.g., batches, of the glycomolecule, having saccharides with the same or similar chemical structure and composition as the preparation having the selected activity. In some embodiments, the method includes further modifying the process to produce a preparation enriched for saccharides with the same or similar chemical structure and composition as the preparation having the selected activity.

In some embodiments, the first and second preparations of glycomolecules differ in glycospecies, e.g., species of the glycomolecule that differ in the chemical structure or composition of the saccharides associated with the glycomolecule, e.g., the presence, absence, position, or number of one or more saccharides. In some embodiments, the first and second preparations can include a number of different glycospecies of the glycomolecule. In some embodiments, the first and second of preparations include a plurality of the same glycospecies at different levels, e.g., the first preparation is enriched for a glycospecies that is not enriched in the second preparation, and/or the second preparation is enriched for a glycospecies that is not enriched in the second preparation. As used herein, the term “enriched for a glycospecies” refers to an increased amount of a glycospecies relative to at least one other glycospecies, and/or relative to the total pool of glycospecies, e.g., a predominant glycospecies.

In another aspect, the invention features molecules prepared by the methods described herein.

In another embodiment, the invention features a method for producing a molecule, e.g., a therapeutic molecule, which includes a first, non-saccharide moiety, e.g., a protein, polypeptide, peptide, amino acid or lipid, and a second, polysaccharide, moiety. The method includes: determining the chemical composition and structure of all or a portion of the second moiety, modifying the structure of the second moiety to provide a modified second moiety, evaluating or screening the molecule having the modified second moiety, e.g., for a biological activity or other chemical or physical property, and attaching the modified second moiety to a different first moiety.

In another aspect, the invention features a method of producing a first molecule which includes a first non-saccharide moiety and a second polysaccharide moiety. The method includes: selecting a modified second moiety which has been modified based upon its ability to confer a desired property on a second molecule, wherein the modified second moiety has been modified based upon its chemical structure; providing the modified second moiety which has been modified based upon its chemical structure and composition; and producing a first molecule which includes a first non-saccharide moiety and the modified second moiety, wherein the modified second moiety alters an activity of the first moiety, to thereby produce a first molecule.

As used herein, a non-saccharide moiety is a chemical moiety which includes a moiety which is other than a saccharide, for example, other than a mono-, di- or polysaccharide. The most preferred non-saccharide moiety is a protein, polypeptide, peptide, amino acid, or lipid. The non-saccharide moiety may contain a saccharide component, for example, a glycoprotein can be a non-saccharide moiety, but as discussed above, the non-saccharide moiety must include an element which is not a saccharide.

### BRIEF DESCRIPTION OF THE DRAWINGS

FIG. 1 is a schematic of methods for rapid sequencing of carbohydrate structures.

FIGS. 2A and 2B are schematics of two techniques for synthesis of modified oligosaccharides. FIG. 2A shows automated solid phase synthesis, and FIG. 2B shows metabolic engineering in cell-based systems.

FIGS. 3A, 3B, 3C and 3D are a set of diagrams depicting notation schemes for branched chain analysis.

FIG. 4 measures the in vivo half-life of anti-MHC antibody (OKT3). 100 μg/kg of purified antibody, either with altered glycosylation or unaltered glycosylation, was injected intravenously into New Zealand rabbits. Blood samples were drawn at selected time points from O-30 hours post-injection. Antibody levels were determined using an IgG-specific ELISA kit.

### DETAILED DESCRIPTION OF THE INVENTION

The invention is based, in part, on the discovery of rapid methods to produce and identify saccharides, and other sugar structures, in order to characterize and develop glycomolecules having altered activities for research and/or therapeutic purposes. The methods include the steps of determining the chemical composition and structure of a saccharide moiety, e.g., a saccharide moiety having or associated with a defined activity, to analyze the sequence of sugars on glycomolecules such as proteins, polypeptides and lipids, modifying the chemical composition or structure of the saccharide moiety, using, for example, enzymatic or solid-phase methods, or metabolic or process engineering, and screening the modified saccharide moiety as part of a glycomolecule, for optimized activity of the glycomolecule. The methods also include enriching a heterogeneous population of glycomolecules for a selected glycospecies.

Saccharides

A polymer as used herein is a compound having a linear and/or branched backbone of chemical units which are secured together by linkages. In some, but not all, cases the backbone of the polymer may be branched. The term “backbone” is given its usual meaning in the field of polymer chemistry. A “chemical unit” as used herein is a chemical compound of carbon, hydrogen, and oxygen in which the atoms of the latter two elements are in the ratio of 2:1. A chemical unit can be, e.g., an aldehyde or ketone derivative of a polyhydric alcohol, particularly of the pentahydric and hexahydric alcohols. The term “saccharide” as used herein refers to one or more chemical units and can include mono, di, tri, poly and heterosaccharides. Examples of monosaccharides include galactose, fucose, sialic acid, mannose, glucose, N-acetylglucosamine (GlcNAc), N-acetylgalactosamine (GalNAc), uronic acid (e.g., glucuronic acid and iduronic acid), xylose, as well as derivatives and analogs thereof. A “polysaccharide” is a biopolymer comprised of two or more linked chemical units. Chemical units of saccharides are much more complex than chemical units of other polymers such as nucleic acids and polypeptides. The saccharide unit has more variables in addition to its basic chemical structure than other chemical units. For example, the saccharide can be acetylated or sulfated at several sites on the chemical unit, or it can be charged or uncharged. In addition, different saccharides can be connected by different glycosidic linkages, and can be branched or linear.

A “plurality of chemical units” is at least two units linked to one another. A substituent, as used herein is an atom or group of atoms that substitute a unit, but are not themselves the units. As used herein with respect to linked units of a polymer, e.g., a polysaccharide, the two units are bound to each other by any physiochemical means. Any linkage, including covalent and non-covalent linkages, is embraced. Naturally occurring linkages are those ordinarily found in nature connecting chemical units of a particular polymer. The chemical units of a polymer can also be linked by synthetic or modified linkages.

The saccharides can be native or naturally occurring saccharides which occur in nature or non-naturally occurring saccharides which do not exist in nature. The saccharides can typically include at least a portion of a naturally occurring saccharides. The saccharides can be isolated or synthesized de novo. For example, the saccharides can be isolated from natural sources, e.g., purified, as by cleavage and gel separation or may be synthesized e.g., by amplification in vitro, synthesized by chemical synthesis, or recombinantly produced, etc.

Methods of Determining Chemical Structure and Compositions of Saccharides

It was discovered that specific chemical properties of a saccharide moiety of a glycomolecule can be identified and manipulated in order to alter an activity, e.g., a therapeutic activity, or decrease or eliminate an activity, e.g., a negative side effect, of the molecule. In addition, the information obtained regarding the manipulated, i.e., modified, saccharide moiety can be applied to other molecules, e.g., other therapeutic molecules. For example, if a modified saccharide moiety is found to have an activity of interest, e.g., increased half-life of a molecule or reduced immunogenicity, that modified saccharide can be formulated (e.g., attached or synthesized) on a different molecule for which that activity, e.g., increased half-life is desired. Conversely, if a modified saccharide moiety or portion thereof is found to have an undesirable activity of interest, e.g., a negative side effect such as increased immunogenicity, that modified polysaccharide or portion thereof can be removed from a different molecule which has that undesirable side effect. The term “glycomolecule” as used herein refers to proteins, polypeptides, peptides and lipids having one or more saccharide moieties associated with it. The term “glycospecies” as used herein refers to species of a glycomolecule that have a particular or unique chemical structure or composition of saccharides associated with the glycomolecule, e.g., the presence, absence, position, or number of one or more saccharides.

The chemical properties of the saccharide can be modified by various techniques in order to alter an activity of active agents (e.g., a non-saccharide moiety of a glycomolecule, e.g., a polypeptide or lipid) associated with the saccharide. In addition, the non-saccharide moiety can be associated with other saccharides in addition to at least one modified saccharide moiety. Methodologies have been developed to determine chemical signatures of saccharides. A chemical signature, as used herein, refers to information regarding, e.g., the identity, mass, charge and number of saccharides, e.g., the mono- and di-saccharide building blocks of a saccharide and the core structure of a branched or unbranched saccharide, information regarding the physiochemical properties such as the overall charge (also referred to as the “net charge”), charge density, molecular size, charge to mass ratio, and sialic acid content as well as the relationships between the mono- and di-saccharide building blocks, e.g., linkages between chemical units of the polysaccharide, branch points, and active sites associated with these building blocks. Information regarding saccharides, e.g., the identity and number of saccharides, e.g., mono- and di-saccharide building blocks, the core structure of a branched polysaccharide, the linkages between chemical units, branch points, sulfonation, sialylation, fucosylation, phosphorylation and acetylation, are considered properties of the chemical structure and composition of a saccharide. As used herein, a chemical signature may refer to all or part of a saccharide. As described herein, it is possible to use specific chemical signatures such as the chemical structure and composition to modify saccharides in order to produce saccharide moieties which alter the activity of the glycomolecules with which they are associated. The chemical signature can be provided by determining one or more primary outputs chosen from the following: the presence or the amount of one or more component saccharides; the presence or the amount of one or more block components, wherein a block component is one made up of more than one saccharides or polysaccharide, the presence of various linkages between chemical units, the presence of different branching structures of a polysaccharide; the presence or amount of one or more saccharide-representative, wherein a saccharide-representative is a saccharide modified to enhance detectability; the presence or amount of an indicator of three dimensional structure or a parameter related to three dimensional structure, e.g., activity, e.g., the presence or amount of a structure produced by cross-linking a saccharide, e.g., the cross-linking of specific saccharides which are not adjacent in the linear sequence; or the presence or amount of one or more modified saccharides, wherein a modified saccharide is one present in a starting material used to make a preparation but which is altered in the production of the preparation, e.g., a saccharide modified by cleavage. The chemical signature can also be provided by determining a secondary output, which include one or more of: total charge and density of charge.

Analysis of a saccharide moiety can be done by constructing a database containing known molecules having known properties, when analyzed using one or more techniques for analysis. A database allows for rapid analysis of saccharide moieties. For example, the known molecules may be saccharides of known composition, structure and molecular mass. The properties may be the data obtained using a technique such as capillary or polyacrylamide gel electrophoresis, high pressure liquid chromatography (HPLC), gel permeation and/or ion exchange chromatography, nuclear magnetic resonance (NMR), mass spectrometry including electrospray or MALDI, modification with an enzyme such as digestion with an exoenzyme or endoenzyme, chemical digestion, or chemical modification. The property may also be measurement of a biological activity, such as the ability to inhibit coagulation, reaction or binding with an antibody, receptor or known ligand, or cleavage by an enzyme with known specificity. The process may be performed for the entire molecule or a portion thereof. The results may also be further quantitated.

Properties to be measured can include one or more of charge, molecular mass, nature and degree of sulfation, phosphorylation or acetylation, and type of saccharide. Additional properties include chirality, nature of substituents, quantity of substituents, molecular size, molecular length, composition ratios of substituents or units, type of basic building block of polysaccharide, hydrophobicity, enzymatic sensitivity, hydrophilicity, secondary structure and conformation (i.e. position of helicies), spatial distribution of substituents, linkages between chemical units, the number of branch points, core structure of a branched polysaccharide, ratio of one set of modifications to another set of modifications (i.e., relative amounts of acetylation or sulfation of various O-positions in sialic acid), and binding sites for proteins.

A property of a saccharide can be identified by means known in the art. Molecular mass, for instance, can be determined by several methods including mass spectrometry. The use of mass spectrometry for determining the molecular mass of polymers is well known in the art. Mass spectrometry has been used as a powerful tool to characterize polymers because of its accuracy (±1 Dalton) in reporting the masses of fragments generated (e.g., by enzymatic cleavage), and also because only picomole sample amounts are required. For example, matrix-assisted laser desorption ionization mass spectrometry (MALDI-MS) has been described for identifying the molecular mass of polysaccharide fragments in publications such as Rhomberg, et al., PNAS USA 95, 4176-4181 (1998); Rhomberg, et al., PNAS USA 95, 12232-12237 (1998); and Ernst, et al. PNAS USA 95, 4182-4187 (1998). Other types of mass spectrometry known the art, such as electron spray-MS, fast atom bombardment mass spectrometry (FAB-MS), gas chromatography/mass spectrometry and collision-activated dissociation mass spectrometry (CAD) can also be used to identify the molecular mass of the polymer or polymer fragments. The compositional ratios of substituents or chemical units (quantity and type of total substituents or chemical units) may be determined using methodology known in the art, such as capillary electrophoresis. A polymer can be subjected to an experimental constraint such as enzymatic or chemical degradation to separate each of the chemical units of the polymers. These units then may be separated using capillary electrophoresis to determine the quantity and type of substituents or chemical units present in the polymer.

The mass spectrometry data may be a valuable tool to ascertain information about the polymer fragment sizes after the polymer has undergone degradation with enzymes or chemicals. After a molecular mass of a saccharide is identified, it can be compared to molecular masses of other known polymers. Because masses obtained from the mass spectrometry data are accurate to one Dalton (1 Da), a size of one or more saccharides obtained by enzymatic digestion may be precisely determined, and a number of substituents (i.e., sulfates and acetate groups present) may be determined. One technique for comparing molecular masses is to generate a mass line and compare the molecular mass of the unknown saccharide to the mass line to determine a subpopulation of saccharide which have the same molecular mass. A “mass line” as used herein is an information database, preferably in the form of a graph or chart which stores information for each possible type of saccharide having a unique sequence based on the molecular mass of the polymer. For instance, a mass line can be generated by uniquely assigning a particular mass to a particular length of a given saccharide (all possible mono- di-, tri-, tetra-, penta-, hexa-, septa-, octa-, up to hexadecasaccharides), and tabulating the results. Methods of generating a database containing such information are provided below.

In addition to molecular mass, other properties may be determined using methods known in the art. The compositional ratios of substituents or chemical units (quantity and type of total substituents or chemical units) can be determined using methodology known in the art, such as capillary electrophoresis. A saccharide can be subjected to an experimental constraint such as enzymatic or chemical degradation to separate each of the chemical units of the saccharide. These units then can be separated using capillary electrophoresis to determine the quantity and type of substituents or chemical units present in the saccharide. Additionally, a number of substituents or chemical units can be determined using calculations based on the molecular mass of the saccharide.

In the method of capillary gel-electrophoresis, reaction samples can be analyzed by small-diameter, gel-filled capillaries. The small diameter of the capillaries (50 microns) allows for efficient dissipation of heat generated during electrophoresis. Thus, high field strengths can be used without excessive Joule heating (400 V/m), lowering the separation time to about 20 minutes per reaction run, therefore increasing resolution over conventional gel electrophoresis. Additionally, many capillaries may be analyzed in parallel, allowing amplification of generated saccharide information.

The saccharide can be further analyzed by applying experimental constraints to the saccharide in a series of repetitions, where the constraints are different for each repetition. The experimental constraints may be any manipulation which alters the saccharide in such a manner that it will be possible to derive structural information about the saccharide or a unit of the saccharide. In some embodiments, the experimental constraint applied to the saccharide can be any one or more of the following constraints: enzymatic digestion, e.g., with an exoenzyme, an endoenzyme, a restriction endonuclease; chemical digestion; chemical modification; interaction with a binding compound; chemical peeling (i.e., removal of a monosaccharide unit); and enzymatic modification, for instance sulfation at a particular position with a sulfotransferase.

The structure and composition of the saccharide moiety can be analyzed, for example, by enzymatic degradation. For each type of monosaccharide and the various types of linkages between a particular monosaccharide and a polysaccharide chain, there exists a modifying enzyme. For example, galactosidases can be used to cleave glycosidic linkages after a galactose. Galactose can be present in a polysaccharide chain through an α1→3 glycosidic linkage or a β1→4 linkage. α-Galactosidase can be used to cleave α1→3 glycosidic linkages after a galactose and β-galactosidase can be used to cleave a β1→4 linkage after a galactose. Sources of β-galactosidase include S. pneumoniae. In addition, various sialidases can be used to specifically cleave an α2→3, an α2→6, an α2→8, or an α2→9 linkage after a sialic acid. For example, sialidase from A. urefaciens cleaves all sialic acids whereas other enzymes show a preference for linkage position. Sialidase (S. pneumoniae) cleaves α2→3 linkages almost exclusively whereas Sialidase II (C. perringens) cleaves α2→3 and α2→6 linkages only. Fucose can be linked to a polysaccharide by any of an α1→2, α1→3, α1→4, and α1→6 glycosidic linkage, and fucosidases which cleave each of these linkages after a fucose can be used. α-Fucosidase II (X. manihotis) cleaves only α1→2 linkages after fucose whereas a-fucosidase from bovine kidney cleaves only α1→6 linkages. GlcNAc can form three different types of linkages with a polysaccharide chain. These are a β1→2, a β1→4 and a β1→6 linkages. Various N-acetylglucosiaminidases can be used to cleave GlcNAc residues in a polysaccharide chain. β-N-Acetylhexosaminidase from Jack Bean can be used to cleave non-reducing terminal β1→2,3,4,6 linked N-acetylglucosamine, and N-acetylgalactosamine from oligosaccharides whereas alpha-N-Acetylgalactosaminidase (Chicken liver) cleaves terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

Enzymes for degrading a saccharide at other specific monosaccharides such as mannose, glucose, xylose and N-acetylgalactosamine (GalNAc) are also known.

Degrading enzymes are also available which can be used to determine branching identity, i.e., is a saccharide mono-, bi-, tri- or tetrantennary. Various endoglycans are available which cleave saccharides having a certain number of branches but do not cleave saccharides having a different number of branches. For example, EndoF2 is an endoglycan that clips only biantennary structures. Thus, it can be used to distinguish biantennary structures from tri- and tetrantennary structures.

In addition, modifying enzymes can be used to determine the presence and number of substituents of a chemical unit. For example, enzymes can be used to determine the absence or presence of sulfates using, e.g., a sulfatase to remove a sulfate group or a sulfotransferase to add a sulfate group.

Glucuronidase and iduronidase can also be used to cleave at the glycosidic linkages after a glucuronic acid and an iduronic acid, respectively. In a similar manner, enzymes exist that cleave galactose residues in a linkage specific manner and enzymes that cleave mannose residues in a linkage specific manner.

The property of the polymer that is detected by this method can be any structural property of a saccharide. For instance, the property of the saccharide may be the molecular mass or length of the saccharide. In other embodiments the property may be the compositional ratios of substituents or units, type of basic building block of a saccharide, hydrophobicity, enzymatic sensitivity, hydrophilicity, secondary structure and conformation (i.e., position of helices), spatial distribution of substituents, linkages between chemical units, number of branch points, core structure of a branched polysaccharide, ratio of one set of modifications to another set of modifications (i.e., relative amounts of sulfation, actylation or phosphorylation at the position for each), and binding sites for proteins.

Methods of identifying other types of properties may be easily identifiable to those of skill in the art and may depend on the type of property and the type of saccharide. For example, hydrophobicity can be determined using reverse-phase high-pressure liquid chromatography (RP-HPLC). Enzymatic sensitivity can be identified by exposing the saccharide to an enzyme and determining a number of fragments present after such exposure. The chirality may be determined using circular dichroism. Protein binding sites may be determined by mass spectrometry, isothermal calorimetry and NMR. Linkages may be determined using NMR and/or capillary electrophoresis. Enzymatic modification (other than degradation) may be determined in a similar manner as enzymatic degradation, i.e., by exposing a substrate to the enzyme and using, e.g., MALDI-MS to determine if the substrate is modified. For example, a sulfotransferase may transfer a sulfate group to an oligosaccharide chain having a concomitant increase of 80 Da. Conformation may be determined by modeling and nuclear magnetic resonance (NMR). The relative amounts of sulfation may be determined by compositional analysis or approximately determined by raman spectroscopy.

Methods for identifying the charge and other properties of saccharides have been described in Venkataraman, G., et al., Science, 286, 537-542 (1999), and U.S. patent applications Ser. Nos. 09/557,997 and 09/558,137, both filed on Apr. 24, 2000, which are hereby incorporated by reference. Other suitable methods for use as described here are known to those skilled in the art. See, for example, Keiser, et al., Nature Medicine 7(1), 1-6 (January 2001); Venkataraman, et al., Science 286, 537-542 (1999). See also, U.S. Pat. Nos. 6,190,522 to Haro, 5,340,453 to Jackson, and 6,048,707 to Klock, for specific techniques that can be utilized.

In addition to being useful for identifying a property, compositional analysis, as described above, also may be used to determine the presence and composition of an impurity as well as a main property of the saccharide. Such determinations can be accomplished if the impurity does not contain an identical composition as the polymer. To determine whether an impurity is present may involve accurately integrating an area under each peak that appears in the electrophoretogram and normalizing the peaks to the smallest of the major peaks. The sum of the normalized peaks should be equal to one or close to being equal to one. If it is not, then one or more impurities are present. Impurities even can be detected in unknown samples if at least one of the disaccharide units of the impurity differs from any disaccharide unit of the unknown. If an impurity is present, one or more aspects of a composition of the components may be determined using capillary electrophoresis.

Database for Determining Chemical Structure and Composition of a Saccharide

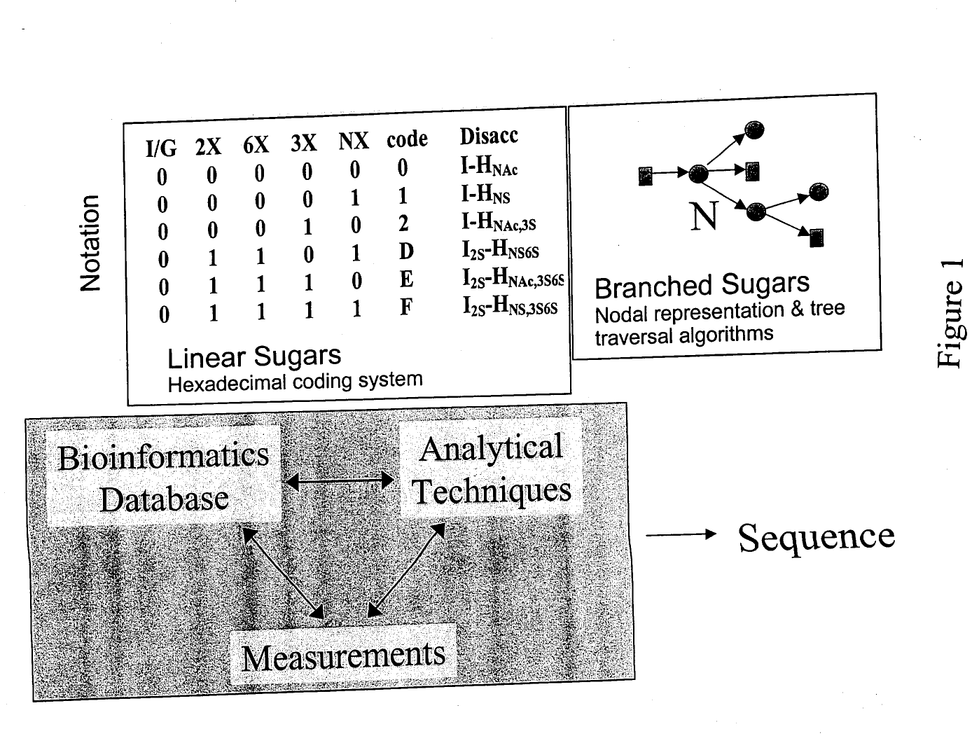

The data obtained using these methods can be analyzed and put into a database (see FIG. 1). A “database,” as used herein, refers to a repository of one or more structures or representatives (unique signatures) of the structure or structures, e.g., mass, charge, mass-to-charge, to which one or more unknown saccharides are compared. The database can be, for example, a flat file, a relational database, a table, an object or structure in a computer readable volatile or non-volatile memory, or any data accessible by computer program. Once the database has been constructed, the saccharide moiety to be characterized, or a portion thereof, can be analyzed, and the results inputted into a computer for comparison with the known polysaccharide molecules in the database. Additional tests can be conducted based on those results, and then, if necessary, the process can be repeated until the saccharide has been identified. For example, the structure and composition of a saccharide can be determined by comparing the length and/or molecular mass of the polysaccharide moiety to a database of saccharides having a known length and/or molecular mass. A subpopulation of saccharides having the same length and/or a similar molecular mass as the saccharide moiety can be selected. An experimental constraint can be applied to the saccharide moiety to determine a property of the saccharide moiety and saccharides of the subpopulation which do not have the same property when the same experimental constraint has been applied to them can be eliminated. Additional experimental constraints can be applied and additional saccharides of the subpopulation can be eliminated based on the results obtained using those additional constraints until the polysaccharide moiety is identified.

A database can be constructed to analyze branched or unbranched polymers, e.g., branched or unbranched polysaccharides.

Branched saccharides include a few building blocks, chemical units, that can be combined in several different ways, thereby, coding for many sequences. For instance, a trisaccharide, in theory, can give rise to over 6 million different sequences. The methods for analyzing branched saccharides, in particular, are advanced by the creation of an efficient nomenclature that is amenable to computational manipulation. Thus, an efficient nomenclature for branched sugars is useful for determining the structure and composition of saccharide moieties. The following are two types of numerical schemes that may be used to encode the sequence information of branched saccharides. These have been developed in order to bridge the widely used graphic (pictorial) representation and the proposed numerical scheme discussed below.

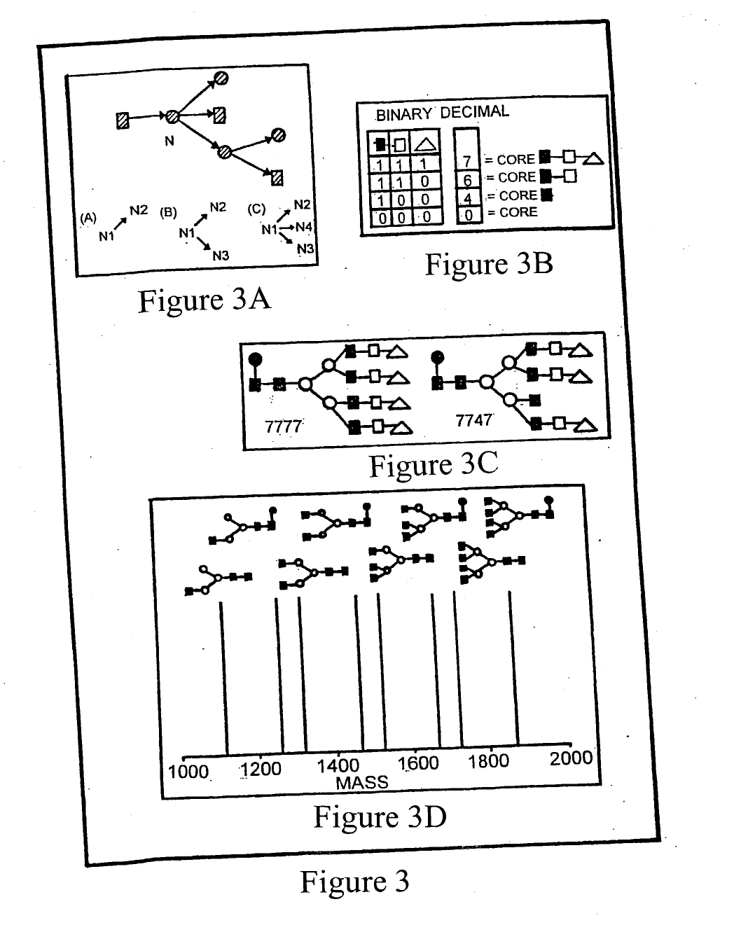

The first notational scheme is a byte-based (binary-scheme) notation scheme. This notation scheme is based on a binary numerical system. The binary representation in conjunction with a tree-traversing algorithm can be used to represent all the possible combinations of the branched saccharides. The nodes (branch points) are easily amenable to computational searching through tree-traversing algorithms (FIG. 3A). FIG. 3A shows a notation scheme for branched sugars. Each monosaccharide unit can be represented as a node (N) in a tree. The building blocks can be defined as either (A), or (B), or (C) where N1, N2, N3, and N4 are individual monosaccharides. Each of these combinations can be coded numerically to represent building blocks of information. By defining glycosylation patterns in this way, there are several tree traversal and searching algorithms in computer science that may be applied to solve this problem.

A simpler version of this notational scheme is shown in FIG. 3B. This simplified version may be extended to include all other possible modifications including unusual structures. For examples, an N-linked glycosylation in vertebrates contains a core region (the tri-mannosyl chitobiose moiety), and up to four branched chains from the core. In addition to the branched chains, the notation scheme also includes other modification (such as addition of fucose to the core, or fucosylation of the GlcNAc in the branches or sialic acid on the branches). Thus, the superfamily of N-linked polysaccharides can be broadly represented by three modular units: a) core region: regular, fucosylated and/or bisected with a GlcNAc, b) number of branches: up to four branched chains (e.g., biantennary, triantennary, tetrantennary), each with GlcNAc, Gal and Neu, and c) modifications of the branch sugars. These modular units may be systematically combined to generate all possible combinations of the saccharide. Representation of the branches and the sequences within the branches can be performed as a n-bit binary code (0 and 1) where n is the number of monosaccharides in the branch. FIG. 3C depicts a binary code containing the entire information regarding the branch. Since there are up to four branches possible, each branch can be represented by a 3-bit binary code, giving a total of 12 binary bits. The first bit represents the presence (binary 1) or absence (binary 0) of the GlcNAc residue adjoining the mannose. The second and the third bit similarly represent the presence or absence of the Gal and the Neu residues in the branch. Hence a complete chain containing GlcNAc-Gal-Neu is represented as binary (111) which is equivalent to decimal 7. Four of the branches can then be represented by a 4 bit decimal code, the first bit of the decimal code for the first branch and the second, the second branch etc. (right).

This simple binary code does not contain the information regarding the linkage (α vs. β and the 1-6 or 1-3, etc.) to the core. This type of notation scheme, however, may be easily expanded to include additional bits for branch modification. For instance, the presence of a 2-6 branched neuraminic acid (Neu) to the GlcNAc in the branch can be encoded by a binary bit.

The second notational scheme that can be used is a prime decimal notation scheme. Similar to the binary notation described above, a second computationally friendly numerical system, which involves the use of a prime number scheme, has been developed. The algebra of prime numbers is extensively used in areas of encoding, cryptography and computational data manipulations. The scheme is based on the theorem that for small numbers, there exists a uniquely definable set of prime divisors. In this way, composition information may be rapidly and accurately analyzed.

This scheme can be illustrated by the following example. The prime numbers 2, 3, 5, 7, 11, 13, 17, 19, and 23 are assigned to nine common building blocks of polysaccharides. The composition of a polysaccharide may then be represented as the product of the prime decimals that represent each of the building blocks. For illustration, GlcNAc is assigned the number 3 and mannose the number 2. The core is represented in this scheme as 2×2×2×3×3=72 (3 mannose and 2 GlcNAcs). This notation, therefore, relies on the mathematical principle that 72 can be only expressed as the combination of three 2s and two 3s. The prime divisors are therefore unique and can encode the composition information.

From this number, the mass of the polysaccharide chain can be determined. The power of the computational approaches of the notional scheme may be used to systematically develop an exhaustive list of all possible combinations of the polysaccharide sequences. For instance, an unconstrained combinatorial list of possible sequences of size mⁿ, where m is the number of building blocks and n is the number of positions in the chain may be used. In FIG. 3C, there are 256 different saccharide combinations that are theoretically possible (4 combinations for each branch and 4 branches=4⁴).

A mass line of the 256 different polysaccharide structures may be plotted. Then, the rules of biosynthetic pathways may be used to further analyze the polysaccharide. In the example (shown in FIG. 3B), it is known that the first step of the biosynthetic pathway is the addition of GlcNAc at the 1-3) linked chain (branch 1). Thus, branch 1 should be present for any of the other branches to exist. Based on this rule, the 256 possible combinations may be reduced using a factorial approach to conclude that the branch 2, 3, and 4 exist if and only if branch one is non-zero. Similar constraints can be incorporated at the notation level before generation of the master list of ensembles. With the notation scheme in place, experimental data can be generated (such as MALDI-MS or CE or chromatography) and those sequences that do not satisfy this data can be eliminated. An iterative procedure therefore enables a rapid convergence to a solution.

To identify branching patterns, a combination of MALDI-MS and CE (or other techniques) can be used. Elimination of the pendant arms of the branched polysaccharide may be achieved by the judicious use of exo and endoenzymes. All antennary groups may be removed, retaining only the GlcNAc moieties extending from the mannose core and forming an “extended” core. In this way, information about branching is retained, but separation and identification of glycoforms is made simpler. One methodology that could be employed to form extended cores for most saccharide structures is the following. Addition of sialidases, and fucosidases will remove capping and branching groups from the arms. Then application of endo-β-galactosidase will cleave the arms to the extended core. For more unusual structures, other exoglycosidases are available, for instance xylases and glucosidases. By addition of a cocktail of degradation enzymes, any polysaccharide moiety can be reduced to its corresponding “extended” core. Examples of degradation enzymes which can be used include galactosidase (e.g., α-galactosidase or β-galactosidase), sialidase, fucosidase, and acetylglucosaminidase. Identification of “extended” core structures can be made by mass spectral analysis. There are unique mass signatures associated with an extended core motif depending on the number of pendant arms (FIG. 3D). FIG. 3D shows a massline of the “extended” core motifs generated upon exhaustive digest of glycan structures by the enzyme cocktail. Shown are the expected masses of mono-, di-, tri- and tetrantennary structures both with and without a fucose linked α1→6 to the core GlcNAc moiety (from left to right). All of the “extended” core structures have a unique mass signature that can be resolved by MALDI-MS (from left to right). Quantification of the various glycan cores present may be completed by capillary electrophoresis, which has proven to be a highly rapid and sensitive means for quantifying polysaccharide structures. See, e.g., Kakehi, K. and S. Honda, Analysis of glycoproteins, glycopeptides and glycoprotein-derived polysaccharides by high-performance capillary electrophoresis. J Chromatogr. A, 1996. 720(1-2):377-393.

Methods for Synthesis or Production of Modified Molecules

Once the starting material has been characterized, and the desired components of the saccharide moiety identified, the modified glycomolecule can be produced.

The method for modifying a saccharide can be determined, e.g., based upon the information obtained regarding the chemical signature of the saccharide. For instance, based upon the structure and composition of the desired saccharide and the nature of the modification, the saccharide can be synthesized, e.g., by enzymatic modification or can be produced by recombinant organisms, e.g., by metabolic engineering, e.g., by controlling degradation or biosynthesis of saccharides. In other embodiments, the modified saccharide can be obtained, e.g., by SAR-based purification methods to obtain a selected saccharide to provide an altered activity to a non-saccharide moiety, e.g., a glycomolecule, e.g., a glycoprotein. In other embodiments, the modified saccharide can be obtained by process engineering, e.g., by manipulating one or more of the parameters of the production and/or manufacturing process, e.g., by altering one or more of the conditions or environment in which recombinant organisms are grown and/or using different host organisms, or by altering purification or concentration methods or formulation.

Enzymatic modification of a polysaccharide moiety can be obtained, e.g., by removing and/or adding select monosaccharides from the polysaccharide. For instance, an enzyme which selectively cleaves a polysaccharide can be used to modify the polysaccharide moiety. Examples of degrading enzymes which can be used include α-galactosidase to cleave a α1→3 glycosidic linkage after a galactose, β-galactosidase to cleave a β1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose. β-N-Acetylhexosaminidase from Jack Bean can be used to cleave non-reducing terminal β1Δ2,3,4,6 linked N-acetylglucosamine, and N-acetylgalactosamine from oligosaccharides whereas alpha-N-Acetylgalactosaminidase (Chicken liver) cleaves terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

In addition, glucuronidase and iduronidase can be used to cleave at the glycosidic linkages after a glucuronic acid and an iduronic acid, respectively.

By selective cleavage, a modified saccharide can be generated such that, e.g., chemical units or regions of the saccharide which are not involved and/or do not influence a desired biological activity can be cleaved, and regions of the saccharide which are involved and/or influence a biological activity remain intact. As used herein, the term “intact” means uncleaved and complete.

Enzymatic modification can also be used, e.g., to add monosaccharides to a polysaccharide. Monosaccharides added to a polysaccharide chain can be incorporated in activated form. Activated monosaccharides, which can be added, include UDP-galactose, UDP-glucose, UDP-N-acetylglucosamine, UDP-N-acetylgactosamine, UDP-Glucuronic acid, UDP-Iduronic acid, UDP-xylose, GDP-mannose, GDP-fucose and CMP-sialic acid. Activated forms of monosaccharides can be generated by methods known in the art. For example, galactose can be activated to UDP-galactose by several ways including: direct phosphorylation at the 1-position to give Gal-1-P, which can react with UTP to give UDP-galactose; Gal-1-P can be converted to UDP-galactose via uridyl transferase exchange reaction with UDP-glucose that displaces Glc-1-P. UDP-glucose can be derived from glucose by converting glucose to Glc-6-P by hexokinase and then either to Fru-6-P by phosphoglucose isomerase or to Glc-1-P by phosphoglucomutase. Reaction of Glc-1-P with UTP forms UDP-glucose. GDP-fucose can be derived from GDP-Man by reduction with CH₂OH at the C-6 position of mannose to a CH₃. This can be done by the sequential action of two enzymes. First, the C-4 mannose of GDP-Man is oxidized to a ketone, GDP-4-dehydro-6-deoxy-mannose, by GDP-Man 4,6-dehydratase along with reduction of NADP to NADPH. The GDP-4-keto-6-deoxymannose is the epimerized at C-3 and C-5 to form GDP-4-keto-6-deoxyglucose and then reduced with NADPH at C-4 to form GDP-fucose. Methods of obtaining other activated monosaccharide forms can be found in, e.g., Varki, A et al., eds., Essentials of Glycobiology, Cold Spring Harbor Press, Cold Spring Harbor, N.Y. (1999).

An activated monosaccharide can be incorporated into a polysaccharide chain using the appropriate glycosyltransferase. For example, to incorporate a sialic acid, CMP-sialic acid onto a polysaccharide chain, a sialyltransferase, e.g., α2→3 sialyltransferase or α2→6 sialyltransferase, can be used. To incorporate a fucose, a fucosyltransferase, e.g., α1→2 fucosyltransferse, α1→3 fucosyltransferase, α1→4 fucosyltransferase or α1→6 fucosyltransferase, can be used. Glycosyltransferases for incorporating galactose and GlcNAc include a galactosyltransferase (e.g., α1→3 galactosyltransferase, β1→4 galactosyltransferase or β1→3 galactosyltransferase) and a N-acetylglucosaminyltransferase (e.g., N-acetylglucosaminyltransferase I, II or III), respectively. Glycosyltransferases for incorporating other monsaccharides are known.

Enzymatic modification of a saccharide can also include both removal of one or more chemical units, e.g., monosaccharides, and then addition of one or more different chemical units, e.g., monosaccharides, to obtain the desired modified saccharide.

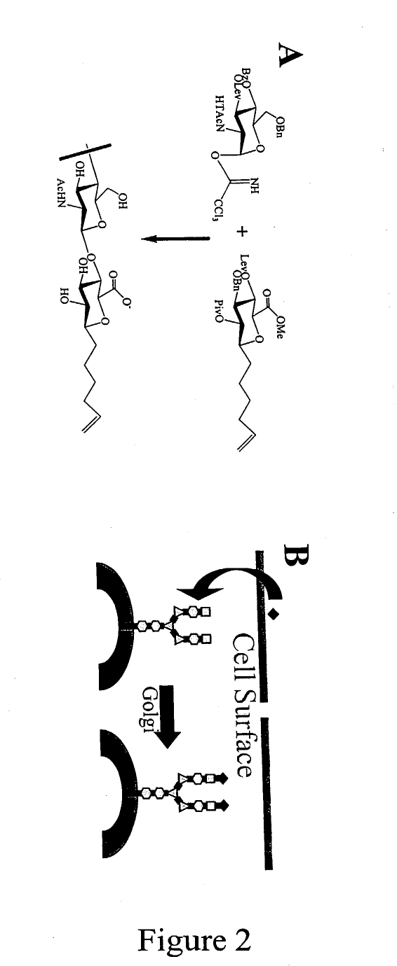

Methods for synthesis using enzymes such as glycosyltransferases are described by Bowman, et al., Biochemistry 40(18):5382-5391 (2001). See also FIG. 2B. Examples of enzymatic synthesis of oligosaccharides are also described in U.S. Pat. No. 6,030,815. U.S. Pat. No. 5,945,322 describes glycosyltransferases for the biosynthesis of oligosaccharides, and genes encoding them. N-containing saccharides and method for the synthesis of N-containing saccharides from amino-deoxy-disaccharides and amino-deoxy-oligosaccharides are described in U.S. Pat. No. 5,856,143. Sialyltransferases are described in U.S. Pat. No. 6,280,989. Keratan sulfate oligosaccharide fraction and pharmaceutical containing the oligosaccharide are described in U.S. Pat. No. 6,159,954.

The methods for synthesis of saccharides include enzymatic as well as chemical synthesis. An example of an automated solid-support synthesis of an oligosaccharide is described by Hewitt and Seeberger, J. Org. Chem. 15:66(12):4233-4243 (June 2001) and Plante, et al., Science 23:291(5508):1523-1527 (2001). This method relies on assembly from monosaccharide units using a solid-phase synthesizer. A branched dodecasaccharide can be synthesized through the use of glycosyl phosphate building blocks and an octenediol functionalized resin. The oligosaccharide is then cleaved from the support. See also, Org. Lett. 2(24):3841-3843 (2000); Andrade, et al., Org. Lett. 1(11):1811-1814 (1999). See further FIG. 2A. An apparatus for the synthesis of saccharide compositions is described in U.S. Pat. No. 6,156,547.

In addition, other saccharides can be synthesized. For instance, lactosamine oligosaccharides and methods for producing lactosamine oligosaccharides are described in U.S. Pat. No. 6,132,994. A lactosamine saccharide can be added to a saccharide chain.

Methods for saccharide characterization and sequencing of oligosaccharides, and methods for reagent array-electrochemical detection are described in U.S. Pat. No. 5,753,454. Methods of sequencing of oligosaccharides are described in U.S. Pat. No. 5,667,984. Methods for determining sugar chain structure are described in U.S. Pat. No. 5,500,342. A process for characterizing the glycosylation of glycoproteins and for the in vitro determination of the bioavailability of glycoproteins are described in U.S. Pat. No. 6,096,555.

Oligosaccharide analogs can also be added to a saccharide. Methods for synthesis of oligosaccharide analogs are well known to those skilled in the art. In general, there are considered to be nine naturally occurring monosaccharides: glucose, xylose, fucose, mannose, N-acetyl galactosamine, N-acetyl glucosamine, galactose, ribose and sialic acid. Any non-natural analogues of these can be added to the glycoproteins. Derivatives, or analogs, of other monosaccharides, i.e. hexose and/or pentose, can also be used. Nonlimiting examples include: amidine, amidrazone and amidoxime derivatives of monosaccharides (U.S. Pat. No. 5,663,355, hereby incorporated by reference), 1,3,4,6-tetra-0-acetyl-N-acylmannosamine or derivative thereof, analogs or derivatives of sugars or amino sugars having 5 or 6 carbons in the glycosyl ring, including aldoses, deoxyaldoses and ketoses without regard for orientation or configuration of the bonds of the asymmetric carbons. This includes such sugars as ribose, arabinose, xylose, lyxose, allose, altrose, glucose, idose, galactose, talose, ribulose, xylulose, psicose, N-acetylglucosamine, N-acetylgalactosamine, N-acetylmannosamine, N-acetylneuraminic acid, fructose, sorbose, tagatose, rhamnose and fucose. Exemplary monosaccharide analogs and derivatives derived from Glc, GlcNAc, Gal, GalNAc, Man, Fuc, and NeuAc as taught in U.S. Pat. No. 5,759,823; hereby incorporated by reference, can be used.

Sialic acids represent the most abundant terminal sugar components on mammalian glycoproteins. Sialic acid/fucose-based pharmaceutical compositions are described in U.S. Pat. No. 5,679,321. Methods for making synthetic ganglioside derivatives are described in U.S. Pat. No. 5,567,684. Bivalent sialyl-derivatized saccharides are described in U.S. Pat. No. 5,559,103. Derivatives and analogues of 2-deoxy-2,3-didehydro-N acetyl neuraminic acid and their use as antiviral agents are reported in U.S. Pat. No. 5,360,817. Examples of preferred sugar monosaccharide analogs include those that functionally mimic sialic acid, but are not recognized by endogenous host cell sialylases. Sialyltransferases and other enzymes that are involved in sialic acid metabolism often recognize “unnatural” or “modified” monosaccharide substrates (Kosa et al., Biochem. Biophys. Res. Commun., 190, 914, 1993; Fitz and Wong, J., Org. Chem., 59, 8279, 1994; Shames et al., Glycobiology, 1, 187, 1991; Sparks et al., Tetrahedron, 49, 1, 1993; Lin et al., J Am. Chem. Soc., 114, 10138, 1992). It has been clearly demonstrated that mannosamine derivatives are converted to sialic acid analogs and incorporated into glycoproteins in cell culture and in rats. In these studies, N-acetylmannosamine (ManNAc), the six carbon precursor for sialic acid, was used as a substrate for the synthesis of metabolically modified glycoproteins, wherein the N-acetyl group of ManNAc was substituted with N-propanoyl, N-butanoyl, or N-pentanoyl (Keppler et al., J. Biol. Chem., 1995, 270,3:1308-1314; and Varki A., J. FASEB, 1991, 2:226-235). Examples of sugar monosaccharide analogs that may also be used include, but are not limited to, N-levulinoyl mannosamine (ManLev), Neu5Acα-methyl glycoside, Neu5Acβ-methyl glycoside, Neu5Acα-benzyl glycoside, Neu5Acβ-benzyl glycoside, Neu5Acα-methylglycoside methyl ester, Neu5Acα-methyl ester, 9-O-Acetyl-N-acetylneuraminic acid, 9-O-Lactyl-N-acetylneuraminic acid, N-azidoacetylmannosamine and O-acetylated variations thereof, and Neu5Acα-ethyl thioglycoside. Examples of sialic acid analogs and methods that may be used to produce such analogs are taught in U.S. Pat. No. 5,759,823 and U.S. Pat. No. 5,712,254; hereby incorporated by reference.

Saccharides can also be produced by metabolic or process engineering in recombinant systems, typically during glycoprotein production. Methods of controlling the degradation of glycoprotein oligosaccharides produced by cultured CHO cells is described by U.S. Pat. No. 5,510,261; methods for controlling sialic acid derivatives in the production of recombinant glycoproteins is described in U.S. Pat. No. 5,459,031. Compounds for altering cell surface sialic acids and methods of use thereof are disclosed in U.S. Pat. No. 6,274,568; methods for sialylation of N-linked glycoproteins expressed in baculovirus expression systems are described in U.S. Pat. No. 6,261,805.

In some aspects, the glycoprotein can be a recombinant glycoprotein produced in a genetically engineered host, either an animal or yeast, fungi, plants, or other eukaryotic cell expression system, although glycoproteins which are normally expressed by the cells can also be modified with non-naturally occurring saccharides. In another embodiment, the non-naturally occurring saccharides are added to the isolated or synthetically produced glycoproteins, by providing the requisite enzymes in combination with the non-naturally occurring substrates, either in a cell-based system or in a cell-free system. The glycoproteins can be modified initially using enzymes to remove all or part of the saccharides, then the non-naturally occurring saccharides added. In yet another embodiment, the starting material may be a protein produced, for example, in a bacterial system wherein the protein is not glycosylated. The protein can then be modified as described herein, to produce a glycoprotein including non-naturally occurring saccharides.

These methods can make use of monosaccharide substrates that are taken up by a host cell, converted to “activated” monosaccharide substrates in vivo and incorporated into the recombinantly expressed protein via the biosynthetic machinery endogenous to the host cell. The protein may be modified by the addition of any monosaccharide, or derivative thereof, that is added to the cell culture, fed to the host animal, and taken up by the host cell where it is attached to the glycoprotein, or which is added to the glycoprotein in a cell-free medium by enzyme(s). The methods are amenable to any host cell which can be manipulated to produce a modified glycoprotein. The host cell uses endogenous biochemical processing pathways to convert, or process, the exogenously added monosaccharide into an activated form that serves as a substrate for conjugation to a target glycoprotein in vivo or in vitro.

The method for altering a saccharide moiety associated with a glycoprotein can includes the following steps: a) contacting a host cell producing the protein to be modified, with a monosaccharide derivative, or analog; and b) incubating the cell under conditions whereby the cell (i) internalizes the monosaccharide derivative, or analog, (ii) biochemically processes the monosaccharide derivative, or analog, and (iii) conjugates the processed monosaccharide derivative, or analog, to an expressed target glycoprotein. The saccharides are added in or administered to a concentration range between 1 micromolar and 100 millimolar, over the course of glycoprotein production or when there is a change in media, depending on culture conditions.

In an in vitro system, the enzymes required for activation and attachment of the saccharides are added to the protein, in the same concentration ranges. The enzymes can be in purified or only partially purified form. Examples of such enzymes are provided herein.

Various systems are available for making these glycoproteins. For example, the glycoproteins can be produced in a cell-based expression system or in a cell-free system. The former is preferred. Cells can be eukaryotic or prokaryotic, as long as the cells provide or have added to them the enzymes to activate and attach the non-natural saccharides and the non-natural saccharides are present in the cell culture medium or fed to the organism including the cells. Examples of eukaryotic cells include yeast, insect, fungi, plant and animal cells, especially mammalian cells, most particularly cells that are maintained in culture such as CHO cells and Green Monkey cells. These organisms all normally glycosylate proteins, although not necessarily in the same manner or with the same saccharides. In the most preferred embodiment, the cells are mammalian. The eukaryotic cells may also be organisms such as animals, where the non-natural saccharides are provided to the animal typically by feeding. In another preferred embodiment, cell lines having genetically modified glycosylation pathways that allow them to carry out a sequence of enzymatic reactions, which mimic the processing of glycoproteins in humans, may also be used.

Currently available systems include but are not limited to: mammalian cells such as Chinese hamster ovary cells (CHO), mouse fibroblast cells, mouse myeloma cells (Arzneimittelforschung. August 1998; 48(8): 870-880), Jurkat cells, HL-60 and HeLa cells; transgenic animals such as goats, sheep, mice and others (Dente Prog. Clin. Biol. 1989 Res. 300: 85-98, Ruther et al., 1988 Cell 53(6): 847-856; Ware, J., et al. 1993 Thrombosis and Haemostasis 69(6): 1194-1194; Cole, E. S., et al. 1994 J. Cell. Biochem. 265-265); plants (for example, Arabidopsis thaliana, rape seed, corn, wheat, rice, tobacco etc.) (Staub, et al. 2000 Nature Biotechnology lS(3): 333-338) (McGarvey, P. B., et al. 1995 Bio-Technology 13(13): 1484-1487; Bardor, M., et al. 1999 Trends in Plant Science 4(9): 376-380); insect cells (for example, Spodoptera frugiperda Sf9, Sf21, Trichoplusia ni, etc. in combination with recombinant baculoviruses such as Autographa californica multiple nuclear polyhedrosis virus which infects lepidopteran cells) (Altmann et al., 1999 Glycoconj. J. 16(2): 109-123); bacteria, including species such as Escherichia coli commonly used to produce recombinant proteins; various yeasts and fungi such as Pichia pastoris, Pichia methanolica, Hansenula polymorpha, and Saccharomyces cerevisiae which have been particularly useful as eukaryotic expression systems, since they are able to grow to high cell densities and/or secrete large quantities of recombinant protein.

Methods of transfecting cells, and reagents such as promoters, markers, signal sequences which can be used for recombinant expression are known.

Non-Saccharide Molecules

The methods described herein can be used to modify a saccharide composition naturally associated with a non-saccharide moiety or can be used to add a saccharide to a non-saccharide moiety that is not naturally associated with the saccharide. In this regard, the non-saccharide moiety can be one that is naturally associated with a different saccharide moiety (e.g., where a saccharide naturally associated with the non-saccharide moiety is replaced with a saccharide which is not naturally associated with the non-saccharide moiety) or the non-saccharide moiety can be one that is not naturally associated with any saccharide moiety. A non-saccharide moiety that is associated with one or more saccharide moieties is referred to herein as a “glycomolecule.” In other aspects, the saccharide moiety can be associated with a non-saccharide moiety at a position in the non-saccharide moiety which is not naturally associated with a saccharide. In some embodiments, the non-saccharide moiety can be associated with more than one saccharide and at least one or more of those saccharides is modified. In other aspects, the non-saccharide moiety can be associated with one or more saccharides, and at least one additional saccharide moiety is added, e.g., at a position in the non-saccharide moiety that is not naturally associated with a saccharide. In yet other embodiments, the non-saccharide moiety can be naturally associated with more than one saccharide, at least one of which has been modified by the methods disclosed herein. In addition, the non-saccharide moiety can have at least one additional saccharide added, e.g., at a position in the non-saccharide moiety that is not naturally associated with a saccharide. A non-saccharide moiety that is associated with one or more saccharide moieties is referred to herein as a “glycomolecule.”

Examples of non-saccharide molecules include, but are not limited to, proteins, polypeptides, peptides, amino acids, lipids, and heterogeneous mixtures thereof.

Proteins or fragments thereof can be associated with one or more modified saccharides to form a glycoprotein or glycopolypeptide using the methods disclosed herein. Examples of classes of proteins which can be used as the non-saccharide portion of a molecule include antibodies, enzymes, growth factors, cytokines and chemokines. Antibodies which can be associated with a modified saccharide, as described herein, include CDP-571, gemtuzumab ozogamicin, biciromab, imciromab, capromab, ¹¹¹indium satumomab pendetide, bevacizumab, ibritumomab tiuxetan, cetuximab, sulesomab, afelimomab, HuMax-CD4, MDX-RA, palivizumab, basiliximab, inolimomab, lerdelimumab, pemtumomab, idiotypic vaccine (CEA), Titan, Leucotropin, etanercept, pexelizumab, alemtuzumab, natalizumab, efalizumab, trastuzumab, epratuzumab, palivizumab, daclizumab, lintuzumab, Cytogam, Engerix-B, Enbrel, Gamimune (IgG), Meningitec, Rituxan, Synagis, Reopro, Herceptin, Sandoglobulin, Menjugate, and BMS-188667. Growth factors, enzymes and receptors which can be used as non-saccharide moieties include Benefix, Meningitec, Refacto, Procit, Epogen, Eprex, Intron A, Neupogen, Humulin, Avonex, Betaseron, Cerezyme, Genotropin, Kogenate, NeoRecormon, Gonal-F, Humalog, NovoSeven, Puregon, Norditropin, Rebif, Nutropin, Activase, Espo, Neupogen, Integrilin, Roferon, Insuman, Serostim, Prolastin, Pulmozyme, Granocyte, Creon, Hetrodin HP, Dasen, Saizen, Leukine, Infergen, Retavase, Proleukin, Regranex, Z-100, somatropin, Humatrope, Nutropin Depot, somatropin, epoetin delta, Eutropin, ranpirnase, infliximab, tifacogin, oprelvekin, interferon-alpha, aldesleukin, OP-1, drotrecogin alfa, tasonermin, oprelvekin, etanercept, afelimomab, daclizumab, thymosin alpha 1, becaplermin, and A-74187. Other non-saccharide moieties which can be used include pexelizumab, anakinra, darbepoetin alfa, insulin glargine, Avonex, alemtuzumab, Leucotropin, Betaseron, aldesleukin, dornase alfa, tenecteplase, oprelvekin, choriogonadotropin alfa, and nasaruplase.

Proteins and fragments thereof can be glycosylated at arginine residues, referred to as N-linked glycosylation, and at serine or threonine residues, referred to as O-linked glycosylation. In some embodiments, the protein or fragment thereof can also be modified. For example, the amino acid sequence of a protein or fragment thereof can be modified to add a site for attaching a saccharide moiety. The amino acid sequence of the protein or fragment thereof can be, e.g., modified to replace an amino acid which does not serve as a site for glycosylation with an amino acid which serves as a site for glycosylation. The amino acid sequence of the protein or fragment thereof can also be modified by replacing an amino acid which serves as a site for one type of glycosylation, e.g., O-linked glycosylation, with an amino acid which serves as a site for a different type of glycosylation, e.g., an N-linked glycosylation. Lastly, an amino acid residue can be added to an amino acid sequence of a protein or fragment thereof to provide a site for attaching a saccharide. An amino acid sequence of a protein or fragment thereof, or the nucleotide sequence encoding it, can be modified by methods known in the art. In particularly preferred embodiments, the protein or fragment thereof is Puregon, Gamimune, Herceptin, NovoSeven, Rebif, Gonal-F, ReoPro, NeoRecormon, Genotropin, Synagis, Cerezyme, Betaseron, Humalog, Engerix-B, Remicade, Enbrel, Rituxan, Avonex, Humulin, Neupogen, Intron A, Epogen and Procit.

In one embodiment, the protein is an erythropoietin-derived protein, e.g., Eprex, Epogen, or human EPO, and one or more of the saccharides associated with human EPO have been replaced by a modified saccharide. For example, human EPO has four glycosylation sites, three N-linked glycosylation sites at residues 24, 38 and 83 of human EPO, and an O-linked glycosylation site at residue 126. One or more of these glycosylation sites in EPO can be analyzed and replaced with a modified saccharide which alters an activity of EPO. In other aspects, human EPO can have a modified saccharide associated with it at a position which does not naturally serve as a glycosylation site in EPO. For example, one, two, three or more saccharides can be associated with EPO at positions not naturally associated with glycosylation in human EPO. EPO has been used to treat patient suffering from anemia, e.g., anemia associated with renal failure, chronic disease, HIV infection, blood loss or cancer. A modified saccharide or saccharides associated with EPO can be screened for various activities including increase half life, increased binding to the EPO receptor, increased stability, altered, e.g., increased, reticulocyte counts.

Methods for the addition of saccharides to protein are known to those skilled in the art. For example, addition of sialyl Lewis acid X to antibodies for targeting purposes is described in U.S. Pat. No. 5,723,583; and modification of oligosaccharides to form vaccines is described in U.S. Pat. No. 5,370,872. A general strategy for forming protein-saccharide conjugates is outlined in U.S. Pat. No. 5,554,730.

Methods for Screening for Altered Activity

Once the modified saccharides have been produced, they can be screened for structure, composition, activity, or pharmacokinetics, and those saccharides having desirable properties selected. The effects of various saccharide modifications can be predicted based upon the structure of the saccharide and the glycomolecule. The chemical signature, e.g., structure and composition, of the modified saccharide can also be determined by the methods described herein and this information can be used to derive a next generation of the glycomolecule with yet another modified saccharide moiety.

Activities which can be screened are those properties affecting the therapeutic utility of molecules, including but not limited to altered clearance, e.g., increased or decreased clearance; altered half-life, e.g., increased or decreased half life; altered stability in vitro (shelf life) or in vivo, e.g., increased stability; altered specificity and/or efficacy (e.g., altered binding or enzymatic activity, e.g., increased or decreased binding or enzymatic activity); altered tissue distribution and targeting, e.g., increased or decreased tissue distribution or targeting; decreased toxicity; decreased immunogenicity; altered pK (e.g., increased pK); altered absorption rate (e.g., increased or decreased absorption rates); altered elimination rate and/or mechanism (e.g., increased or decreased elimination rates); and altered bioavailability (e.g., increased bioavailability). In addition, the following activities can be screened for: specific binding to biomolecules (for example, receptor ligands); hormonal activity; cytokine activity; inhibition of biological activity or interactions of other biomolecules (for example, agonists and antagonists of receptor binding); enzymatic activity; anti-cancer activity (anti-proliferation, cytotoxicity, antimetastasis); immunomodulation (immunosuppressive activity, immunostimulatory activity); anti-infective activity; antibiotic activity; antiviral activity; anti-parasitic; anti-fungal activity; and trophic activity.

The activity can be measured and detected using appropriate techniques and assays known in the art. Antibody reactivity, e.g., immunogenicity, and T-cell activation can be considered bioactivities. Bioactivity can also be assessed in vivo where appropriate. This can be the most accurate assessment of the presence of a useful level of the bioactivity of interest. Enzymatic activity can be measured and detected using appropriate techniques and assays known in the art. Proteins and fragments thereof have been shown to influence the autophosphorylation of receptors in vitro, by assaying the amount of radiolabeled phosphate retained by the receptor before and after interaction with the protein. This can be shown using standard techniques. By influencing the phosphorylation of cell surface receptors the isolated proteins and fragments thereof can directly influence the activity of the cellular processes these receptors control. Methods to allow post translational, or peptide modification, of the proteins or fragments thereof in vitro are known. Such modifications include, but are not limited to, acylation, methylation, phosphorylation, sulfation, prenylation, further glycosylation, carboxylation, ubiquitination, amidation, oxidation, hydroxylation, adding a seleno-group to amino acid side chains (for example, selenocysteine), and fluorescent labeling.

Further in vitro analyses are used to study the effects of the glycomolecules on cell viability. For example, proteins or fragments thereof that either interrupt, stimulate, or decrease vital cellular processes may be used to infect cells, such as tumor cells, in culture. Once infected, cell growth and viability is analyzed by methods known in the art.

In vivo analyses using animal models can be used to determine the effects of a glycomolecule within an intact system. For example, in the field of immunology, glycomolecules such as glycoproteins or fragments thereof can be administered to an animal and its peripheral blood monocytes can be used in the generation of antibodies directed against the protein. To detect changes in immunogenicity, animal models can also be used; in one example, the modified glycoprotein or fragments thereof is administered to an animal, e.g., a mouse, and the animal is monitored for the generation of antibodies against the glycoprotein. Computer models known in the art may also be used to evaluate changes in immunogenicity, e.g., as described in Renouf and Hounsell, Adv Exp Med Biol 376:37-45 (1995).

In the case of viral proteins for use with, for example, viral vectors, therapeutic viruses, and viral capsid delivery compositions, desired characteristics to be retained can include the ability to assemble into a viral particle or capsid and the ability to infect or enter cells. Such characteristics are useful where the delivery properties of the viral proteins are of interest, or as applied to use of the components as immunogens in vaccines.

Stability of a glycomolecule may be measured both by in vivo and in vitro techniques well known in the art. For example, blood samples may be drawn, from a host animal, at selected timepoints and antibody levels monitored and determined using ELISA kits available in the art.

In addition, other methods of screening for altered activities of a glycomolecule are well known to those skilled in the art. For example, glycoform fractions of recombinant soluble complement receptor 1 (sCR1) screened for extended half-lives in vivo are described in U.S. Pat. No. 5,456,909. In addition, antibodies having modified carbohydrate content and methods for preparation and use are described by U.S. Pat. No. 6,218,149.

### EXAMPLES

Protein Production

For each of the examples listed below, both an IgG antibody (humanized IgG4 in CHO or IgG1 in a hybridoma cell line) and erythropoietin are used as representative glycoproteins. The culturing of the cell lines is completed under sterile conditions using aseptic technique. Hybridoma or CHO cells are grown in T225 flasks from Gibco BRL in media of the following composition: 500 mls GIBCO/Invitrogen Iscove's modified media containing 10 mls 7.5% Sodium Bicarbonate, 50 mls Fetal Calf Serum (low IgG-containing), and 5 mls Glutamine/Penicillin/Streptomycin.

Cell lines (either IgG or erythropoietin producing) are split every 48-72 hours or when they appeared confluent. To complete this, the media is removed and the flask is flushed with sterile phosphate buffered saline (15 mls) to remove any media components. 2 mL of warmed Trypsin/EDTA is added to the flask to remove the adherent cells from the plastic. Once removed (˜1 min), 10 mLs of fresh media is added and the cell suspension is transferred to a conical tube and centrifuged at 1000 rpm for 5 min. The supernatant is vacuum-aspirated and fresh media is added to resuspend the cell pellet which is aliquoted into new flasks and allowed to grow. 500 mL-1 L of media containing recombinant protein is then subjected to purification as outlined below.

Protein Purification

Antibodies obtained from either CHO cells or hybridomas are purified using a protein A column (Amersham Pharmacia Biotech). Prior to column purification, the conditioned media is 0.2 μm filtered and the pH is adjusted to 7.0. The column is primed using 5 column volumes of “load” buffer (50 mM sodium phosphate, 500 mM NaCl pH 7.8), 3 column volumes of “elution” buffer (100 mM Glycine pH 3.0), and finally 5 column volumes of load buffer. Conditioned media is added to the column such that ˜10 mg of IgG is loaded per ml of resin. Then the column is washed with 5 column volumes of load buffer prior to addition of 5 column volumes of elution buffer. After elution, the protein is immediately brought to pH 7.0 using 1M Tris pH 9.0.

Human erythropoietin (EPO) is expressed as a 6×-His tagged fusion protein in an appropriate vector such as pcDNA 3.1 (Invitrogen). Conditioned media containing the His-tagged protein is 0.2 μm filtered. Prior to purification, the following buffers are run over the chelating resin. Five column volumes of “binding” buffer: 20 mM Na Phosphate, 500 mM NaCl, 5 mM Imidazole, pH 7.9, then 3 column volumes of “charge” buffer: 200 mM nickel sulfate. The column is then washed with 5 column volumes of binding buffer, the material is applied, the column washed with binding buffer, and the protein is eluted with a high imidazole buffer (20 mM Na Phosphate, 500 mM NaCl, 500 mM Imidazole, pH 7.9). Purity and amount of the proteins are assessed by silver stain gel and the micro BCA assay (BioRad).

Analysis of Glycan Structure

Glycan structures after modification are analyzed by MALDI mass spectrometry. Prior to analysis, glycan structures are typically harvested from the purified protein. Typically, 100 μg of purified glycoprotein (IgG or EPO) is digested at 37° C. for 4 hrs in a 0.1 M sodium phosphate pH 7.5 buffer containing 0.5% SDS, 1% β-mercaptoethanol, 1% NP-40 and 1000U of PNGase F (from New England Biolabs). The released glycan is purified using an activated carbon cartridge (Glyko, Inc.), eluted in 30% acetonitrile, dried and redissolved in HPLC-grade water prior to analysis.

MALDI analysis is completed on a Voyager DE STR system (Applied Biosystems) using an accelerating voltage of 22 kV. Analysis in the positive and negative modes are completed using either a 1:1 mixture of 20 mg/mL DHB in acetonitrile and a 25 mM aqueous solution of spermine or a saturated solution of 2,4,6-trihydroxyacetophenone (THAP, Fluka Chemicals) in 30% acetonitrile.

Measurement of Serum Half-Life

Increasing amounts of recombinant glycoprotein (10-500 μg) is injected i.v. via the tail vein. At time intervals ranging from 0 hr-48 hr., 100 μL of blood is withdrawn. The serum is separated via centrifugation at 1800×g for 10 minutes and analyzed using a sandwich ELISA (ZeptoMetrix, Inc.) format. Results are plotted as amount of protein vs. time after administration. Half-life is calculated using a non-compartment model.

### Example 1

Fractionation of Glycan Isoforms

Chromatographic separation of recombinantly produced protein can be completed to isolate in a preparative manner a particular glycan isoform. Either IgG or EPO (1 mg) in 10 mM sodium phosphate pH 6.7 is added to a NucleoPac™ PA100 column (Dionex) at a flow rate of 1.5 mL/min and a 60 minute gradient of 0-50% of 0.3 M ammonium acetate pH 6.7 was completed. Fractions are collected, and the glycan structure is isolated and analyzed as described above.

Enzymatic Modification of Recombinant Protein Ligands

The following examples use ex vivo modification of glycan structures, after purification.

### Example 2

Adding of Galactose to N-linked Sugar Structures

To 10 mg/mL of purified IgG or EPO in 50 mM Tris, 0.15M NaCl, 0.05% NaN₃ is added 100 mU/mL of β1→4 galactosyltransferase. The solution is incubated with 5 mM UDP-galactose, 10 mM MnCl₂ at 37° C. for 24-48 hrs. Incorporation is measured by taking an aliquot of the reaction mixture, isolating the glycan structure and analyzing using the MALDI procedure outlined above.

### Example 3

Sialic Acid Capping

The glycoprotein (either modified as in example 2 or otherwise) is dissolved at 10 mg/mL in 50 mM Tris, 0.15M NaCl, 0.05% NaN₃. The solution is then incubated with 5 mM CMP-sialic acid and 100 mU/mL α2→3 (or α2→6) sialyltransferase at 32° C. for 2 days. The degree of incorporation is measured using the isolation and MALDI procedure outlined above.

### Example 4

Addition of Other Branches

In some cases, it is desirable to increase the branching of a glycan structure, via the addition of a core α1→6 fucose or the addition of β1→4-N-acetylglucosamine. In these cases, modification is accomplished essentially the same as above. To 10 mg/mL of purified IgG or erythropoietin in 50 mM Tris, 0.15M NaCl, 0.05% NaN₃ is added 100 mU/mL of either α1→6 fucosyltransferase or β1→4-N-acetylglucosaminyltransferase III. The solution is then incubated with 5 mM of the activated sugar at 37° C. for 24-48 hrs. Incorporation is then measured by isolating the glycan structure and analyzing it using MALDI-MS.

### Example 5

Metabolic Engineering

Synthesis of Modified Monosaccharide (ManProp):

To mannosamine hydrochloride in methanol is added 1 eq. of sodium methoxide (0.5M in methanol) and the mixture is allowed to stir for 1 hr. Then 1.1 molar equivalents of propionic anhydride is added and the mixture is allowed to stand for 3-5 hrs until the reaction is complete. The solvent is then removed via vacuum prior to peracetylation.

In these cases, peracetylated monosaccharides have been shown to passively diffuse through mammalian cell membranes and undergo subsequent deacetylation by intracellular esterases, allowing efficient incorporation into proteins of modified monosaccharides. To peracetylate the monosaccharide ManProp, 100 mM acetic anhydride is added to 200 mM ManProp in pyridine, and the reaction is allowed to stir for 4 hrs. The solvent is removed and the residue is re-dissolved in methylene chloride, washed with water and dried. The resulting material (Ac₄ManProp) is purified using silica gel chromatography and analyzed using FAB MS and ¹H NMR.

Incorporation of Modified Monosaccharide:

To CHO cells in media is added a 100 mM ethanolic solution of the Ac₄ManProp such that the concentration of the modified monosaccharide in the media is 50-300 μM. The cells are allowed to grow to confluence and fresh monosaccharide is added with every splitting. Incorporation of the modified monosaccharide is measured after purification of the recombinant protein using MALDI-MS as described above.

To increase the level of uptake of the metabolic precursor, several parameters were varied. First, addition of cytidine, a necessary precursor of CMP-sialic acid, at concentrations of 1-10 mM, increases the level of incorporation as measured by MALDI-MS. In addition, disabling the enzyme UDP-N-acetylglucosamine 2-epimerase results in an increase in the amount of incorporation of the modified monosaccharide. Synthetic monosaccharides likely compete with the physiological precursor N-acetylmannosamine and its metabolic products for the sialic acid machinery, resulting in only moderate expression of modified sialic acid derivatives on the surface of recombinant glycoproteins. Thus, a cell lacking this enzyme can only generate sialic acid moieties through a scavenge pathway, i.e. modified monosaccharides added to the media, resulting in a larger degree of incorporation. This enzyme can be disabled by methods commonly known in the art.

Finally, incorporation of modified sialic acid monosaccharide analogues can be increased via the addition of a glycosyltransferase, such as β1→4 galactosyltransferase or α2→3 (or α2→6) sialyltransferase. To transfect CHO cells producing recombinant protein, the Lipofectamine™ 2000 protocol from Invitrogen is followed. In this case, CHO cells are seeded at 0.5×10⁵ cells per well in a 24 well plate one day before transformations are carried out so that the cells would be roughly 90% confluent on the day of transformation. Transformations are done in triplicate for 2 clones containing the erythropoietin gene in a PCDNA3.1 vector from Invitrogen. Fifty μl of F10 media is mixed with 0.8-1 μg of DNA. In a separate tube, 50 μl of F10 media is mixed with 2-3 μl of lipofectamine. Mixtures are incubated at room temperature for 5 minutes, mixed together, and incubated for an additional 20 minutes at room temperature. Each DNA-lipofectamine mixture is then added to one well of the 24 well plate. After 4 hours of incubation at 37° C., the media is removed from the wells and replaced with fresh media. Twenty-four hours after transformation, the media is replaced with selective media containing 500 μg/ml Geneticin® (purchased from Invitrogen). Cells are grown for several days, and media was harvested to assay for erythropoietin using an ELISA kit. Protein expression of the relevant transferase is confirmed, and cell populations are expanded and clonal populations were established.

### Example 6

Glyco-Modification of an Anti-MHC Antibody

A hybridoma cell line expressing an anti-MHC antibody (OKT3) was grown in roller bottles in Iscove's modified Dulbecco's medium containing 10% Ultra-low IgG fetal bovine serum (Gibco). 1,3,4,6,-tetra-O-acetyl-N acylmannosamine, or derivative thereof, to a final concentration of 10-50 μM. The cells were allowed to grow with fluid renewal every 2-3 days and at these time points, antibody was harvested from the spent media.

Media containing the antibody was run over a protein A column (Sepharose CL4B fast flow) to purify the antibody. Bound antibody was washed with ice cold PBS and 10 mM Tris pH 8.0. following the washing steps, the antibody was eluted with 100 mM glycine pH3 and immediately brought to pH 7.0 with 1M Tris. Antibody purity and concentration were assessed by denaturing a portion of the preparation and running a silver stain gel as well as determining the A₂₈₀ (OD₂₈₀ of l=0.75 mg/ml).

To assess the glycosylation pattern of the OKT antibody, 100 μg of the preparation was denatured and digested with PNGase F overnight at 37° C. After digestion, the glycan was purified via an activated charcoal column. Glycoforms were assessed by capillary electrophoresis using a 50 mM phosphate pH 2.5 running buffer and/or via MALDI mass spectrometry using a aqueous saturated solution of DHB matrix containing 300 mM spermine-HCL.

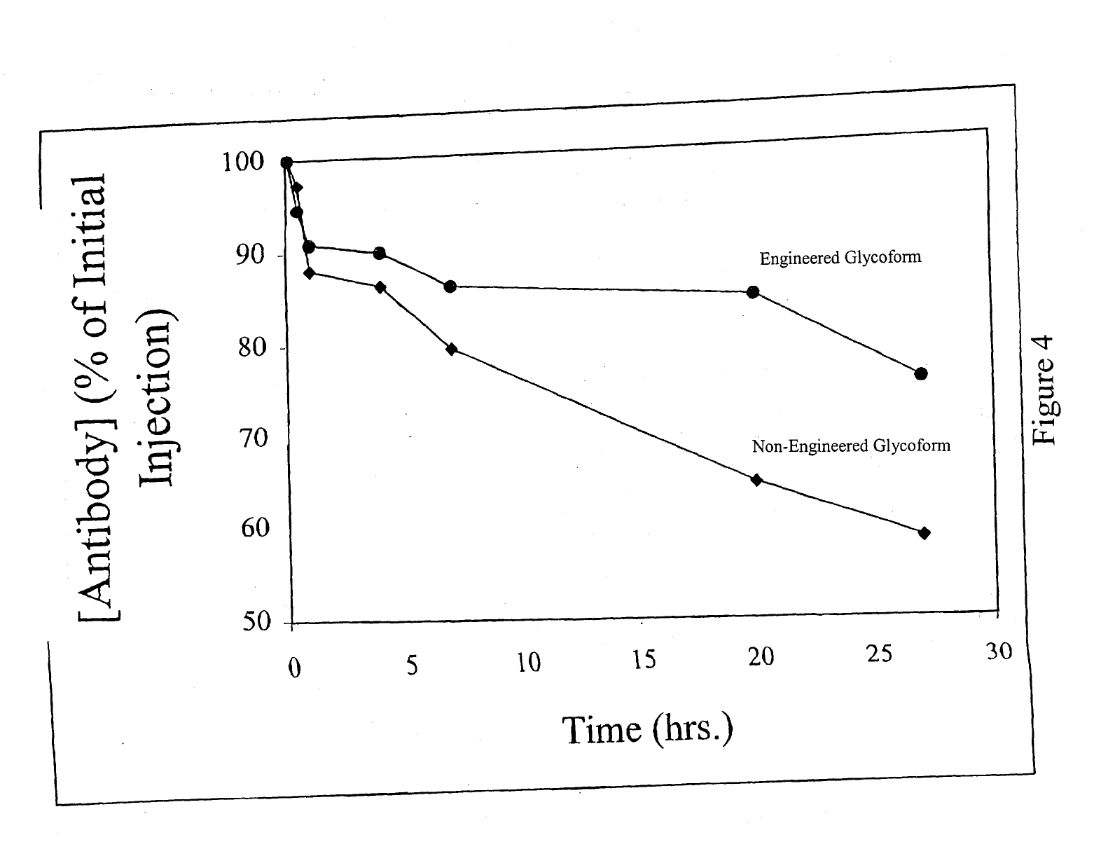

In order to assess the in vivo half-life of the glyco-modified antibody, 100 μg/kg purified antibody was injected intravenously into New Zealand rabbits. Blood samples were drawn at selected time points from 0-100 hours post-injection. Antibody levels were determined using an IgG-specific ELISA kit (FIG. 4).

The references, patents and patent applications cited herein are incorporated by reference. Modifications and variations of these methods and products thereof will be obvious to those skilled in the art from the foregoing detailed description and are intended to be encompassed within the scope of the appended claims.

We claim:

1. A method of selecting a preparation of a glycomolecule having one or more saccharides which are correlated with a selected activity, the method comprising:
providing a plurality of preparations of a glycomolecule;
determining the chemical structure or composition of one or more saccharides associated with the glycomolecules in each of the plurality of preparations; and
selecting a preparation of glycomolecules having one or more saccharides which are correlated with the selected activity.

2. The method of claim 1, wherein the glycomolecule is a glycoprotein.

3. The method of claim 1 wherein the selected activity is an altered therapeutic efficacy or therapeutic profile.

4. The method of claim 3, wherein the altered therapeutic profile comprises reduced immunogenicity.

5. The method of claim 1, wherein the plurality of preparations comprises two or more preparations, each preparation is produced using a different process than the other preparation.

6. The method of claim 5, wherein the plurality of preparations comprises three or more preparations, and each preparation is produces by a different process than the others.

7. The method of claim 5, wherein the processes differ in one or more experimental or manufacturing parameters.

8. The method of claim 1, further comprising analyzing one or more of the preparations of the glycomolecule to evaluate whether the one or more preparations has the selected activity.

9. The method of claim 1, wherein the chemical structure or composition of the glycomolecule in one of the preparations differs from the chemical structure or composition of the glycomolecule of another of the preparations, and the preparation that includes the glycomolecule having the selected one or more saccharides that are correlated with the selected activity, is selected.

10. The method of claim 9, wherein the chemical composition or structure of the one or more saccharides of the glycomolecule of the selected preparation is further used as a standard for evaluating additional preparations of the glycomolecule.

11. A method of selecting a preparation of a glycomolecule having one or more selected activities, the method comprising:
providing a plurality of preparations of a glycomolecule produced by different processes;
determining the chemical structure or composition of one or more saccharides associated with the glycomolecule in each of the plurality of preparations;
analyzing one or more of the preparations of the glycomolecule to evaluate the presence, absence, or level of a selected activity;
correlating the chemical structure or composition of one or more saccharides associated with the glycomolecule from the plurality of preparations with the presence, absence, or level of the activity; and
selecting a preparation having the selected activity.

12. The method of claim 11, further comprising selecting the process which produced the selected preparation, and performing the selected process to make additional preparations of the glycomolecule.

13. The method of claim 1, wherein at least one of the preparations of glycomolecule differs from at least one of the other preparations in that one or more of the saccharides of the glycomolecule is different.

14. The method of claim 1, wherein at least one of the preparations of glycomolecule comprises different glycospecies from at least one of the other preparations.

15. The method of claim 1, wherein at least one of the preparations of glycomolecules comprises a heterogeneous population of glycospecies.

16. The method of claim 15, wherein the heterogeneous population of glycospecies comprises a population of glycomolecules having the same non-saccharide moiety and different saccharide moieties.

17. The method of claim 1, wherein two or more of the preparations comprise a number of different glycospecies of the glycomolecule.

18. The method of claim 1, wherein two or more of the preparations comprise a plurality of the same glycospecies but at least one of the preparations comprises the plurality of the same glycospecies, but at least one glycospecies at different levels than at least one of the other preparations.

19. The method of claim 1, wherein at least one or more of the plurality of preparations is enriched for a glycospecies that at least one or more of the other preparations is not enriched for the glycospecies.

20. The method of claim 19, wherein the preparation enriched for a glycospecies having one or more saccharides is correlated with the selected activity, and the preparation enriched for the glycospecies is selected.

21. The method of claim 19, wherein the preparation that is not enriched for a glycospecies having one or more saccharides is correlated with the selected activity, and the preparation which is not enriched for the glycospecies is selected.

22. A method of selecting a preparation of a glycomolecule having a selected activity, the method comprising:
providing a first preparation of a glycomolecule made by a first process;
providing a second preparation of the glycomolecule made by a second process;
evaluating each of the first and second preparation for a selected activity; and
determining the chemical structure or composition of one or more saccharide associated with the glycomolecule in each of the first and second preparations.

23. The method of claim 22, wherein the second process is different from the first process in one or more experimental or manufacturing parameters

24. The method of claim 12, wherein the selected activity is altered therapeutic efficacy or therapeutic profile.

25. The method of claim 24, wherein the altered therapeutic profile is the presence or absence of a side effect.

26. The method of claim 26, wherein the side effect is immunogenicity.

27. The method of claim 22, further comprising selecting the preparation having or lacking the selected activity.

28. The method of claim 22, wherein the first preparation has a different level of the selected activity than the second preparation.

29. The method of claim 28, wherein the different level of activity is the absence or presence of the activity.

30. The method of claim 28, wherein the chemical structure or composition of one or more saccharides of the glycomolecule of the first preparation differs from the chemical structure or composition of the second preparation.

31. The method of claim 30, further comprising correlating the difference in one or more saccharides of the glycomolecule of the first and second preparations with the difference in the selected activity of the first and second preparations.

32. The method of claim 31, wherein the first preparation has the selected activity, and the first preparation is selected.

33. The method of claim 32, wherein the first process is selected.

34. The method of claim 33, wherein the chemical structure or composition of the first preparation is selected as a reference standard for one or more preparations made by a process other than the first and second processes.

35. The method of claim 22, further comprising selecting the first or second process which produced a preparation having a desired activity, and performing the selected process to make additional preparations of the glycomolecule.

36. The method of claim 22, wherein the first and second preparations of glycomolecules comprise different glycospecies.

37. The method of claim 22, wherein the preparations include a number of different glycospecies of the glycomolecule.

38. The method of claim 22, wherein the plurality of preparations include a plurality of the same glycospecies at different levels.

39. The method of claim 22, wherein each of the plurality of preparations is enriched for a glycospecies that is not enriched in at least one other preparation.
